(19) United States
(12) Patent Application Publication
(10) Pub. No.: US 2002/0028764 A1
(43) Pub. Date: Mar. 7, 2002

(54) Treatment of acute and chronic liver disease

(75) Inventors: Thorbjorn Grofte, Viby J. (DK); Hendrik Vilstrup, Risskov (DK)

Correspondence Address: BROWDY AND NEIMARK, P.L.L.C., 624 Ninth Street, N.W., Washington, DC, 20001, US

(73) Assignee: Aarhus Amt., Hojbjerg (DK)

(21) Appl. No.: 09/928,832

(22) Filed: Aug. 14, 2001

Related U.S. Application Data
(60) Provisional application No. 60/237,715, filed on Oct. 5, 2000.

(30) Foreign Application Priority Data
Sep. 4, 2000 (DK) PA 2000 01317

(51) Int. Cl. A01N 37/18; A61K 38/00; C07K 5/00; C07K 7/00; C07K 16/00; C07K 17/00

(52) U.S. Cl. 514/2; 514/12; 530/324

(57) ABSTRACT

The present invention relates to IGF-1 treatment of an individual, such as e.g. a human being, suffering from an acute or chronic liver disease including hepatic cirrhosis. Acute and chronic liver disease according to the invention are characterized by low circulating IGF-1 and IGFBP3 levels. According to one preferred embodiment of the present invention, IGF-1 is administrered to a human being subcutaneously, preferably in the thigh or the abdominal skin, and preferably in two daily doses of about 50 microgram/kg twice a day. The present invention demonstrates that this dosis regime is able to restore normal IGF-1 levels in patients with liver cirrhosis, and the dose is well-tolerated by the patients.

### TECHNICAL FIELD OF THE INVENTION

The invention relates to a method for treatment of acute and chronic liver disease in an individual. The treatment involves administration of insulin growth-like factor 1 (IGF-1) to an individual in need thereof. The liver disease may occur in combination with other diseases such as e.g. diabetes mellitus.

### BACKGROUND OF THE INVENTION

Growth factors are growth promoting peptides that stimulate a wide variety of biological responses in a defined population of target cells. A variety of growth factors have been identified, including transforming growth factor beta (TGF-β), epidermal growth factor (EGF), platelet-derived growth factor (PDGF), fibroblast growth factor (FGF), and insulin-like growth factor 1 (IGF-1).

IGF-1 is a naturally occurring growth promoting peptide and shares considerable structural and functional homology with insulin and is synthesized in the liver. It consist of 70 amino acids in a single poly-peptide chain with homology to pro-insulin and has a molecular weight of approximately 7.5 kilodaltons (kD).

IGF-1 in the circulation is bound to 6 different binding proteins (IGFBP-1-6), which binds 95% of total IGF-1, a major fraction of this is bound to IGFBP3. About 5% is free IGF-1 that acts through both its own receptors and insulin receptors. The biological effects are to some degree qualitatively similar to the action of insulin.

IGF-1 induces cellular growth and differentiation, stimulates cellular glucose uptake and metabolism and exerts strong anabolic action on protein metabolism. IGF-1 has been studied in a variety of human conditions, including Laron dwafism, acute renal failure, and AIDS, and the safety profile of the drug when given subcutaneously is well-defined.

IGF-1 and IGFBP-3 may be purified from natural sources or produced by recombinant means. For instance, purification of IGF-1 from human serum is well known to the art (Rinderknecht et al., 1976, Proc. Natl. Acad. Sci, (USA) 73: 2365-2369). Production of IGF-1 by recombinant processes is shown in EP 0,128,733, published in December of 1984. IGFBP-3 may be purified from natural sources using a process such as that shown in Baxter et al., (1986, “Growth Hormone-Dependent Insulin-Like Growth Factors (IGF) Binding Protein from Human Plasma Differs from Other Human IGF Binding Proteins”, Biochem Biophys. Res, Comm, 139: 1256-1261). IGFBP-3 may be synthesized by recombinant organisms as discussed in Sommer et al. (1991, “Molecular Genetics and Action of Recombinant Insulin-Like Growth Factor Binding Protein-3”, in Modem Concepts of Insulin-Like Growth Factors, E. M. Spencer, ed., Elsevier, N.Y., pp. 715-728).

Hepatocytes are believed to be the major source of circulating IGF-1 and IGFBP3 and the synthesis is under influence of growth hormone (GH). IGF-1 circulates in the blood bound to IGF-1 binding proteins (IGFBP's) and interacts with specific receptors on target tissue, such as liver and muscle. The majority of IGFBP-1 and IGFBP-3 is also believed to be produced in the liver.

IGF-1 mediates the major effects of growth hormone, and is thus a primary mediator of growth after birth. IGF-1 has also been implicated in the actions of various other growth factors as treatment of cells with such growth factors leads to increased production of IGF-1. IGF-1 has insulin-like activities and are mitogenic (stimulate cell division) for the cells in neural tissue, muscle, reproductive tissue, skeletal tissue and a wide variety of other tissues.

Unlike most growth factors, the IGF-1 is present in substantial quantity in the circulation, but as most circulating IGF-1 is bound to IGF-1 binding protein 3 (IGFBP-3), only a very small fraction of this IGF is free in the circulation or in other body fluids. IGF-1 may be measured in blood serum to diagnose abnormal growth-related conditions, e.g., pituitary gigantism, acromegaly, dwarfism, various growth hormone deficiencies, etc. Although IGF-1 is produced in many tissues, most circulating IGF-1 is believed to be synthesized in the liver.

Almost all IGF-1 circulates in a non-covalently associated ternary complex composed of IGF-1, IGFBP-3, and a larger protein subunit termed the acid labile subunit (ALS). This ternary complex is composed of equimolar amounts of each of the three components. ALS has probably no direct IGF binding activity and appears to bind only to the IGF/IGFBP-3 binary complex. The ternary complex comprising IGF, IGFBP-3 and ALS has a molecular weight of approximately 150 Kd.

This ternary complex has been alleged to function in the circulation “as a reservoir and a buffer for IGF-1 that prevents rapid changes in the concentration of free IGF” (Blum et al., 1991, “Plasma IGFBP-3 Levels as Clinical Indicators” in Modem Concepts in Insulin-Like Growth Factors, E. M. Spencer, ed., Elsevier, N.Y., pp. 381-393).

The ternary complex is also believed to play an important role in the prevention of hypoglycemia due to high doses of IGFI, by binding IGF-1/IGFBP-3 complex and restricting its distribution (Zapf et al., 1994, “Intravenously Injected Insulin-like Growth Factor (IGF) I/IGF Binding Protein-3 Complex Exerts Insulin-like Effects in Hypophysectomized, but Not in Normal Rats”, Clinical Investigation 95: 179-186). ALS is growth hormone-dependent, so hypophysectomized rats and other subjects with insufficient levels of growth hormone have little to noALS (Baxter, 1990, 1990, “Circulating Levels and Molecular Distribution of the Acid-labile (.alpha.) Subunit of the High Molecular Weight Insulin-like Growth Factor-Binding Protein Complex” J Clin. Endocrinol. 70(5): 1347-1353).

As nearly all of the IGF-1 and IGFBP-3 in the circulation is in complexes, very little free IGF can be detected. Moreover, a high level of free IGF in blood is undesirable. High blood levels of free IGF lead to serious hypoglycemia, due to the insulin-like activities of IGF, as well as other adverse side effects. In contrast to IGF-1 and IGFBP-3, there appears to be a substantial pool of free ALS in plasma which most likely forms a ternary complex with any IGF/IGFB-3 complex entering the circulation. However, it has been hypothesised that saturating free ALS by administration of high levels of IGF-1/IGFBP-3 also leads to hypoglycemia (Zapf et al., ibid).

Although IGFBP-3 is the most abundant IGF binding protein in the circulation, at least five other distinct IGF binding proteins (IGFBPs) have so far been identified in various tissues and body fluids. Although these proteins bind IGF-1, they each originate from separate genes and have distinct amino acid sequences. Thus, IGF-1 binding proteins are not merely analogs or derivatives of a common precursor. Unlike IGFBP-3, the other IGFBPs in the circulation are not saturated with IGF-1. None of the IGFBPs other than IGFBP-3 are apparently able to form the 150 Kd ternary complex with IGF-1 and ALS.

The literature has ascribed to IGFBP-3 both a passive role as a carrier of IGF-1 extending its circulatory half-life and an active role as a promoter of IGF-1 activity. For example, it has been disclosed by BioGrowth, Inc. that IGFBP-3 significantly accelerates healing in an animal wound-healing model and that the complex of IGF-1 and IGFBP-3 stimulates cortical and trabecular bone growth in rats in preliminary experiments, suggesting that the BP may be useful in treating osteoporosis. See Bioventure View, Vol. IV, No. 1 (Jan. 31, 1989), pages 19-20. See also EP 294,021 and EP 375,438 disclosing use of IGFBP-3 in conjunction with IGF-1 to treat diseases such as osteoporosis and human GH deficiency, and to heal wounds and increase animal growth, including delivery to bony tissues to stimulate bone growth (e.g., p. 8 of EP 294,021 and p. 11 of EP 375,438, in addition to WO 90/00569). However, no data are provided for these speculative uses. Talkington-Verser (Proceed. Intern. Symp. Control. Rel. Bioact. Mater. (1989), vol 16: 223-224) has suggested that IGFBP-3 (termed IGF-CP) may increase IGF-directed bone growth in rats. However, also in this case neither protocols nor experimental data were provided.

Several reviews cast further doubt on the precise biological activities of the IGFBPs. For example, Zapf et al. (Growth Factors: From Genes to Clinical Application, Karolinska Nobel Conference Series, Eds. Vicki Sara et al., (Raven Press 1990), p. 227) states that inhibitory as well as enhancing effects of IGF carrier proteins on IGF actions have been observed in vitro (citing: DeMellow et al., Biochem. Biophys. Res. Comm., 156: 199-204 (1988); Elgin et al., Proc. Natl. Acad. Sci. USA, 84: 3254-3258 (1987); Knauer and Smith, Proc. Natl. Acad. Sci. USA, 77: 7252-7256 (1980); Meuli et al., Diabetologia, 14: 255-259 (1978); Schweiwiller et al., Nature, 323: 169-171 (1986). Zapf et al. further state that it is still unknown whether the different IGFBP species known thus far differ with respect to inhibiting or enhancing the biological effects of IGF.

On page 241 of the same book, Hall et al. state that, in general, IGFBP-1, similar to IGFBP-3, is found to inhibit IGF-1 stimulation of amino acid uptake and DNA synthesis, citing, inter alia, Walton et al., P.S.E.B.M., 190: 315-319 (1989).

Baxter (Comp. Biochem. Physiol., 91B, 229-235 (1988), p. 232-233) stated that despite an increasing interest in IGFBPs, their functions are still poorly understood. Baxter points to some evidence that association with BPs may not always inhibit the activity of the IGFs and that cell types producing the BPs might be able to enhance their IGF responsiveness in an autocrine fashion. Examples cited are that some high molecular weight complexes from human plasma retain biological activity in rat adipocyte assays for insulin-like activity, cultured human fibroblasts secrete a BP of 35 kD that increases cell IGF binding, and a pure preparation of amniotic fluid BP significantly potentiates the effect of IGF-1 in stimulating DNA synthesis in porcine smooth muscle cells and fibroblasts from various species. Furthermore, it has been shown that IGFBP-3 blocks the hypoglycemic action of IGF-1 when administered subcutaneously together with the IGF-1 in a 1:1 ratio (Spencer et al., 2nd International Symposium on Insulin-Like Growth Factors/Somatomedins, Jan. 12-16, 1991, program and Abstracts p. 269).

In another hypothesis it has been suggested that IGFBPs are produced locally in all tissues to maintain locally produced IGF-1 near cells requiring the IGF-1, reducing the active role of IGF-1 bound to BPs and IGF-1 circulating in the blood (Isaksson et al., Endocrine Reviews (1987), vol. 8: 426-438). It has also been reported that IGF-1 is produced locally in bone (Nilsson et al., Science (1986), vol. 233: 571-574; Nilsson et al., J. Endocr. (1989), vol. 122: 69-77.

Furthermore, work by Conover (Prog. Abstract 186 presented at the 72nd Annual Meeting of Endocrine Society (June 1990)) reported in vitro data suggesting that the activity of the IGFBPs in enhancing the activity of IGF-1 is dependent on cells being exposed to the BPs alone. No response to IGF-1 was observed in cells incubated with pre-mixed BP and IGF-1. If the BP ifself was initially incubated with the cells and followed by addition of IGF-1, the activity of the added IGF-1 was enhanced. These data suggest that co-mixing IGF and a IGFBP and co-injecting the complex would not result in an enhancement of the activity of the IGF-1.

The below-listed patents and -patent applications disclose various methods of treatment involving administration of IGF-1. Treatment of acute and chronic liver disease, including cirrhosis, as described herein is not disclosed.

WO 99/24062 (Chiron Corp.) relates in one aspect to a highly concentrated IGF-1 composition comprising biologically active IGF-1 in a concentration of at least 250 mg/ml. Also disclosed is a suggested method of therapy for an IGF-1 responsive condition in an individual. The method comprises administration to the individual of a pharmaceutical composition comprising a highly concentrated form of biologically active IGF-1 present in a concentration of at least about 250 mg/ml. Hepatic cirrhosis appears along with acute and chronic liver failure among a large number of conditions that are hypothesised as being responsive to IGF-1. The authors suggest treating liver cirrhosis in an individual by administration to the individual of pharmaceutical compositions comprising at least about 250 mg/ml IGF-1. Treatment of acute and chronic liver disease including cirrhosis in an individual involving administration to the individual of IGF in pharmaceutically effective daily amounts of about 100 μg to 250 μg per kg body weight is not disclosed.

U.S. Pat. No. 6,034,059 (Chiron Corp.) relates to a method for the treatment of a catabolic state in a patient and involves administration to the patient of IGF-1 in conjunction with a hypocaloric diet. It is reported that administration of IGF-1 will be useful in treating conditions such as chronic bowel disease, e.g. Crohns disease, protein losing enteropathies, short gut syndromes, postgastroenteritic malabsorption states, cystic fibrosis, chronic or acute pancreatitis, and hepatitis. As acute or chronic liver disease will induce GH resistance, IGF-1 in combination with a hypocaloric diet may be particularly valuable in acute hepatic failure, where protein loading can be dangerous, and in catabolic conditions associated with chronic liver disease. Treatment of acute and chronic liver disease including cirrhosis in an individual involving administration of IGF to the individual is not disclosed.

U.S. Pat. No. 5,492,891 (Novo Nordisk A/S) relates to a method for treating an individual with alcoholic cirrhosis of the liver and consequently very low concentrations of IGF-1 in the blood, in spite of increased growth hormone concentrations. The treatment comprises periodically injecting the individual with human growth hormone in order to increase the level of IGF-1 in the blood of the individual. Treatment of acute and chronic liver disease including cirrhosis in an individual involving administration of IGF to the individual is not disclosed.

U.S. Pat. No. 5,824,642 (Genentech, Inc.) relates to a method for increasing the growth rate of a human patient having partial growth hormone insensitivity syndrome, but not Laron syndrome. The patient is characterized as having i) a height of less than about −2 standard deviations below normal for age and sex, ii) a serum level of high-affinity growth hormone binding protein that is at least 2 standard deviations below normal levels, iii) a serum level of IGF-1 that is below normal mean levels, and iv) a serum level of growth hormone that is at least normal. One method according to the invention comprises administering an effective dose of a growth hormone to a patient. Also disclosed is a treatment with an effective amount of IGF-1 in the absence of growth hormone administration. Treatment of acute and chronic liver disease including cirrhosis in an individual involving administration of IGF to the individual is not disclosed.

U.S. Pat. No. 5,681,814 (Genentech, Inc.) relates to a formulation for IGF-1 that is useful in treating hyperglycemic disorders and, in combination with growth hormone, in enhancing growth of a mammal. It is disclosed that IGF-1 is a polypeptide naturally occurring in human body fluids, and that especially the liver produces IGF-1 together with specific IGF-binding proteins. IGF-1 has been isolated from human serum and produced recombin antly (EP 123,228 and EP 128,733). IGF-1 is a potent anabolic protein (Tanner et al., Acta Endocrinol., 84: 681-696 (1977); Uthne et al., J. Clin. Endocrinol. Metab., 39: 548-554 (1974)). An intravenous bolus injection of IGF-1 is known to lower blood glucose levels in humans (Guler et al., N. Engl. J. Med., 317: 137-140 (1987), and IGF-1 also promotes growth in several metabolic conditions characterized by low IGF-1 levels, such as i) hypophysectomized rats (Guler et al., Endocrinology, 118: Supp 129 abstract; Skottner et al., J. Endocr., 112: 123-132 (1987); Guler et al., Proc. Natl. Acad. Sci. USA, 85: 4889-4893 (1988); Froesch et al., in Endocrinology, Intl. Congress Series 655, ed. by Labrie and Proulx (Amsterdam: Excerpta Medica, 1984), p. 475-479!, ii) diabetic rats (Scheiwiller et al., Nature, 323: 169-171 (1986)), and iii) dwarf rats (Skottner et al., Endocrinology, 124: 2519-2526 (1989)). The kidney weight of hypophysectomized rats increases substantially upon prolonged infusions of IGF-1 subcutaneously (Guler et al., Proceedings of the 1st European Congress of Endocrinology, 103: abstract 12-390 (Copenhagen, 1987)). IGF-1 is also known to improve glomerular filtration and renal plasma flow in human patients (EP 327,503 published Aug. 9, 1989; Guler et al., Proc. Natl. Acad. Sci. USA, 86: 2868-2872 (1989)). The IGF-1 formulation of the invention is suggested to be useful for treatment of any condition that would benefit from treatment with IGF-1, including, for example, diabetes, chronic and acute renal disorders, such as chronic renal insufficiency, necrosis, etc., obesity, hyperinsulinemia, GH-1nsufficiency, Turner's syndrome, short stature, undesirable symptoms associated with aging such as increasing lean mass to fat ratios, immuno-deficiencies including increasing CD4 counts and increasing immune tolerance, catabolic states associated with wasting, etc., Laron dwarfism, insulin resistance, and the like. The IGF-1 formulation also has an increased potency in treating humans with hyperglycemic disorders—including all forms of diabetes, such as type I and type II diabetes, as well as hyperinsulinemia and hyperlipidemia—by reducing their glucose levels. Treatment of acute and chronic liver disease including cirrhosis in an individual involving administration of IGF to the individual is not disclosed.

U.S. Pat. No. 5,273,961 (Genentech, Inc.) relates to a method for prophylactic treatment of mammals at risk for acute renal failure, whether due to renal ischemia or nephrotoxic damage. The method involves administering to the mammal, before or at the time that the acute renal failure is expected to occur or is occurring, an effective amount of IGF-1. Initiation of IGF administration after acute renal damage is expected to occur or has occurred is disclaimed. However, the administration of IGF-1 may be continued after acute renal damage is expected to occur or is occurring. Acute renal failure in the form of ischemic renal injury or nephrotoxic damage is particularly mentioned. IGF-1 may be administered together with any one or more of its binding proteins, i.e. any one or more of IGFBP-1, IGFBP 2, IGFBP-3, IGFBP-4, IGFBP-5, or IGFBP-6. The preferred binding protein for IGF-1 is IGFBP-3 (disclosed in WO 89/09268 and by Martin and Baxter, J. Biol. Chem., 261: 8754-8760 (1986)). The glycosylated IGFBP-3 protein forms part of a glycoprotein complex found in human plasma that carries most of the endogenous IGFs. It is also regulated by growth hormone. Renal tissue is described as being very responsive to IGF-1 due to high concentrations of IGF-1 receptors on renal cell membranes (Hammerman, Am J. Physiol., 257: F503-F514 (1989); Rogers and Hammerman, Proc. Natl. Acad. Sci. USA. 86: 6363-6366 (1989); Hammerman and Gavin, Am. J. Physiol., 251: E32-E41 (1986); Pillion et al., Am. J. Physiol., 255: E504-E512 (1988); Hammerman and Rogers, Am. J. Physiol., 253: F841-F847 (1987)). It is further disclosed that elevated levels of circulating GH is associated with increased renal plasma flow and glomerular renal flow. Measures of renal hemodynamics rise within several hours after a single injection of growth hormone, at about the same time that serum IGF-1 concentrations increase. In particular, IGF-1 is reported to i) increase glomerular filtration and renal plasma flow (Guler et al., Proc. Natl. Acad. Sci. USA, 86: 2868-2872 [1989]), and ii) stimulate renal phosphate transport and plasma 1,25-dihydroxyvitamin D₃ (Caverzacio et al., Endocrinol., 127: 453-459 (1990)). Also, a short term infusion of IGF-1 alone into rats fasted for 60-72 hours was found to increase glomerular filtration rate (Hirschberg and Koppel, J. Clin. Invest, 83: 326-330 (1989); Hirschberg et al., J. Clin. Invest, 87: 1200-1206 (1991)). Administration of IGF-1 to humans is also reported to elevate glomerular filtration rate and renal plasma flow (Guler et al., Acta Endocrinol., 121: 101-106 (1989); Froesch et al., Trends in Endocrinology and Metabolism, p. 254-260 Vol. 1, Issue 5 (Elsevier Science Pub. Co., 1990); U.S. Pat. No. 5,106,832). Treatment of acute and chronic liver disease including cirrhosis in an individual involving administration of IGF to the individual is not disclosed.

U.S. Pat. No. 5,187,151 (Genentech, Inc.) relates to a method for producing an anabolic state in a mammal by co-administration of a combination of effective amounts of IGF-1 and an IGF binding protein in a defined molar ratio in the absence of growth hormone. The aim is to produce a greater anabolic response in the mammal than that achieved when using IGF-1 alone in an amount equal to that used for IGF-1 in the combination. The method involves co-administering the IGF-1 and IGFBP in a molar ratio of IGFBP to IGF-1 of about 0.5:1 to 3:1 by subcutaneous (sc) bolus injection. The IGF-1 and IGFBP mixture to be used in the therapy will be formulated and dosed in a fashion consistent with good medical practice, taking into account, among others, the particular growth defect or catabolic state to be corrected, and the particular IGFBP being utilized. Treatment of acute and chronic liver disease including cirrhosis in an individual involving administration of IGF to the individual is not disclosed.

U.S. Pat. No. 5,948,757 (Celtrix Pharmaceuticals, Inc.) relates to a method for providing high dose IGF-1 therapy by administering a complex of IGF-1 and IGFBP-3. The IGF-1/IGFBP-3 complex is reported to be administered in unexpectedly high doses without inducing IGF-1-related side effects. It is desirable to give high dose IGF-1 therapy by administering IGF-1/IGFBP-3 complex to an individual because of the increased efficacy achieved. Among the many indications suggested to be responding to IGF treatment is acute and chronic renal disorders as well as catabolic conditions arising e.g. from trauma, severe burns, and major surgery (Clemmons and Underwood (1994, “Uses of Human Insulin-like Growth Factor-1 in Clinical Conditions” J Clin. Endocrinol. Metabol. 79(1): 4-6). Also, Miller et al. reported on the effects of IGF-1 on renal function in end-stage chronic renal failure (Miller et al. (1994), Kidney International 46:201-207). It is stated that conditions allegedly responding to high dose IGF-1 therapy are those generally responding to administration of IGF-1. According to the patent, such conditions include, but are not limited to: neurological disorders such as amyotrophic lateral sclerosis, Charcot-Marie-Tooth Syndrome, diabetic neuropathy, and drug-1nduced neuropathy (such as peripheral neuropathy induced by chemotherapeutic agents including vincristine, cisplatin, and the like), and pulmonary disorders such as chronic obstructive pulmonary disease; renal disorders such as glomerulonephritis, glomerulosclerosis, interstitial nephritis, acute tubular necrosis, diabetic nephropathy, autoimmune nephropathy, and acute and chronic renal failure; growth disorders such as growth hormone insufficiency, hypopituitarism, growth hormone resistance and Laron dwarfism; recovery from bodily insults, such as recovery from trauma, burns, bone fractures or surgery; gastrointestinal disorders such as short bowel syndrome and pancreatic disease; reversal of catabolism in subjects with acquired immune deficiency syndrome (AIDS), cancer cachexia, or steroid-1nduced catabolism (such as can occur as a result of long term steroid therapy for disorders such as asthma, autoimmune disease, inflammatory bowel disease, immune suppression for organ transplantation, and rheumatoid diseases); bone disorders such as osteoporosis, osteopetrosis, osteogenesis imperfecta, and Paget's disease; reproductive disorders such as hypogonadotropic hypogonadism, male infertility, failure of gamete maturation, and polycystic ovarian disease; and hematopoietic disorders such-as anemia, plasma cell dyscrasias, erythropoietin insensitivity and deficient total hemoglobin. Treatment of acute and chronic liver disease including cirrhosis in an individual involving administration of IGF to the individual is not disclosed.

U.S. Pat. No. 5,723,441 (Celtrix Pharmaceuticals, Inc.) relates to a treatment with IGF/IGFBP-3 complex that increases renal tubular mass and potentiates and/or stimulates kidney function in subjects suffering from acute and chronic renal failure or renal insufficiency resulting from disorders such as glomerulonephritis, glomerulosclerosis, interstitial nephritis, acute tubular necrosis due to ischemia and drug-1nduced toxicity, diabetic and autoimmune nephropathies and renal dysfunction due to acute and chronic rejection episodes in post-transplantation patients. The main patent claim is limited to a method of treating diabetic nephropathy or autoimmune nephropathy in an individual. Conditions responding to treatment with IGF are alleged to include acute or chronic renal failure, resulting from diabetes, ischemia, drug induced toxicity, post-transplantation rejection with or without the need for dialysis; glomerulonephritis; glomerulosclerosis; interstitial nephritis; and acute tubular necrosis. Patients may have physical findings such as anuria, lethargy, coma and decreased general growth rate. Indicative laboratory results include increased plasma levels of creatinine, urea and uric acid (BUN), proteinuria, decreased GFR, RPF and renal size as determined by urogram, altered acid/base balance and changes in urine specific gravity. Treatment of acute and chronic liver disease including cirrhosis in an individual involving administration of IGF to the individual is not disclosed.

U.S. Pat. No. 6,071,880 (Dalhousie Univeristy) relates to a method for the treatment of chronic renal insufficiencies and related chronic indications in mammals, employing IGF-1 as the active agent. IGF-1 is reported to be an effective agent for enhancing kidney development in an individual suffering from chronic organ injury. Examples of a renal insufficiency include renal dysplasia, renal hypoplasia, congenital renal anomaly, and acute renal failure. There are also provided methods to enhance kidney development in individuals suffering from chronic organ injury. Individuals for whom enhanced kidney development is indicated include adults who have undergone transplantation of a small kidney (wherein further growth of the organ is ablated), subjects who suffer from renal tubule poisoning, and subjects who have undergone chemotherapy, such as e.g. cancer patients. Treatment of acute and chronic liver disease including cirrhosis in an individual involving administration of IGF to the individual is not disclosed.

U.S. Pat. No. 5,783,559 (Pharmacia & Upjohn AB) relates to a stable, isotonic, injectable and long term stable solution consisting essentially of IGF-1 and a phosphate buffer in an amount of from 5 to 20 mmol and having a pH of from 5.5 to 6.5. Treatment of acute and chronic liver disease including cirrhosis in an individual involving administration of IGF to the individual is not disclosed.

U.S. Pat. No. 5,756,463 (Pharmacia & Upjohn AB) relates to a method for counteracting i) a decrease in nitrogen balance and ii) a decrease in protein synthesis in an individual, and the method comprises administration to an individual of a combination of insulin and IGF-1. In particular the invention is directed to a treatment of one or more of i) protein catabolism due to glucocortoid excess, ii) cardiac disease, iii) insulin-resistance, and iv) liver disease. The individual in question may have a high serum level of IGFBP1 or a high serum level of low molecular weight binding proteins. Treatment of acute and chronic liver disease including cirrhosis in an individual involving administration of IGF to the individual is not disclosed.

U.S. Pat. No. 5,466,670 (Pharmacia AB) relates to the use of IGF-1 for the manufacture of a medicament for treatment of Type 1 diabetes mellitus. The medicament comprises a subcutaneous dose not greater than needed to achieve an IGF-1 serum level characteristic for healthy individuals, i.e. a physiological replacement of serum IGF-1. It is stated that the invention provides a physiological restoration of circulating IGF-1 levels and gives reduced growth hormone levels through a feed back mechanism. This normalisation of the levels of growth hormone and IGF-1 leads to an increased sensivity for insulin and to a reduction in the rapid increase in the morning blood glucose levels seen in type 1 diabetics.

It is reported that all subjects participating in the studies that generated the results on which the invention is based had a diabetes duration of at least 5 years (range 5-10 years), and all subjects were in good health with normal hepatic and renal function. In a disclosure of the background art it is noted that large doses of hIGF-1 lower blood glucose in non-diabetic animals and humans (Zapf J. et al. J Clin-1nvest, Jun Vol:77 (6) (1986) 1768-75 and Guler H-P et al, N Engl. J. Med, 317 (1987) 137-140). The glucose lowering effect of IGF-1 is reported to be mediated primarily by an increased glucose uptake, while glucose production rates are unchanged. The relative paucity of IGF-1 receptors in adult liver may explain this finding (Caro J. F. et al., J. Clin. Invest, 81 (1988) 976-981). It is stated that it is likely that the effects of IGF-1 are largely mediated through muscle. Similar distinctions in the distribution of receptors are thought to explain the less potent antilipolytic effects of IGF-1 as compared to insulin both in vitro (Bolinder et al, J. Clin. Endocrinol. Metab, 65, (1987) 732-737) and in vivo (Zapf J. et al. 1986, Guler H-P et al. 1987, Giacca A et al. 1990). IGF-1 is also described as leading to a decreased proteolysis and reduced amino acid levels in non-diabetic rats (Jacob R. et al., J Clin. Invest, 83, (1989) 1717-1723). Treatment of acute and chronic liver disease including cirrhosis in an individual involving administration of IGF to the individual is not disclosed.

U.S. Pat. No. 5,434,134 (Pharmac IA AB) relates to a method for increasing cardiac muscle protein synthesis and for treating cardiomyopathies, acute heart failure or acute insult. In particular the invention is directed to a method for prevention of cardiomyopathies following drug administration, inflammation, infection, sepsis or ischaemia. Treatment of acute and chronic liver disease including cirrhosis in an individual involving administration of IGF to the individual is not disclosed.

U.S. Pat. No. 5,068,224 (KabiVitrum AB) relates to a method for improving the regeneration of transected peripheral nerves in an individual by administering a sufficient amount of insulin-like growth factor 1 (IGF-1) to the individual. Treatment of acute and chronic liver disease including cirrhosis in an individual involving administration of IGF to the individual is not disclosed.

Several investigators have examined the effects of IGF-1 in rats with cirrhosis induced by repeated administration of carbon tetrachloride (a hepatotoxin) and Phenobarbital (upregulates hepatic enzymes which metabolise carbon tetrachloride, thereby producing more severe and reproducible hepatic injury). In this model, IGF-1 in low dose a) reverses insulin resistance as assessed by glucose clamp studies (Petersen 1997), b) increased food intake, efficiency of food utilisation, and improves nitrogen balance (Picardi 1997), c) improves intestinal absorption of galactose (Castilla-Cortazar 1997), d) improves biochemical markers of liver function (e.g. serum albumin concentration) and reduces hepatic fibrosis when administrated concomitantly with tetrachloride/Phenobarbital and after induction of cirrhosis (Castilla-Cortazar 1997). Pascual have reported an altered intestinal transport of amino acids in cirrhotic rats and studied the effect of insulin-like growth factor-1: Am J Physiol Gastrointest Liver Physiol. 2000 August; 279(2):G319-24).

In each case the analysed rats had acquired—within a short period of time—an experimentally (chemically) induced cirrhosis. Such a model is incompatible with clinical reality, neither with respect to analysed species—humans versus rodents—nor with respect to the disease in question, as human cirrhosis develops over a period of years, mostly due to alchohol abuse, whereas the artificially generated cirrhosis in rats has been induced over a few weeks by exposing the rats to extremely high doses of a chemical toxin. It is a fact that both histologically and clinically there are very significant differences between experimental cirrhosis in animals and liver cirrhosis in humans.

The difficulties experienced by the skilled artisan wishing to evaluate histologically and clinically any disease caused by different means in different species is consequently quite overwhelming. This is also demonstrated by the fact that experienced medical researchers would not attempt to perform a direct extrapolation of results obtained from an animal study to results obtained from treating a different kind of species—such as human beings. The skilled artisan would recognise that an animal study, and in particular a study involving rodents, differ quantitatively, qualitatively and clinically compared to a study of humans.

The skilled artisan will also be aware that one primary purpose of using animals in medical research is that such research may—at the most—generate a mere hypothesis capable of being further investigated in a study of humans. The fact that only a hypothesis can be established is supported by data from the pharmaceutical industry according to which more than 90% of all clinical trials do not enter clinical phase 3 in spite of promising results obtained from animal studies. This clearly demonstrates how difficult it is to extrapolate results from animal studies to the clinical realities of the real world involving medical treatment of human beings.

The complexities of liver disease are evident from a study of Caregaro (Nutritional and prognostic significance of insulin-like growth factor 1 in patients with liver cirrhosis: Nutrition. 1997March; 13(3):185-90), who concluded that a reduction of IGF-1 in connection with liver disease is probably caused by multiple factors, most of which are related to the severity of the disease in question. Furthermore, the action of IGF-1 is modulated by several binding proteins which are themselves subject to a complex pattern of regulation.

Castilla-Cortazar (1997, ibid.) reported that although subcutaneous administration of very low doses of IGF-1 (20 microgram (μg) IGF-1 per kilogram body weight per day) did not result in any overall increase in overall levels of IGF-1, subcutane administration of IGF-1 in the above-mentioned amounts induced significant changes in the pattern of IGF binding proteins acting as critical modulators of the biological actions of IGF-1. Similarly low doses of IGF-1 (20 μg IGF-1 per kilogram body weight per day) have also been administered subcutaneously by Picardi (ibid.) and Castilla-Cortazar (2000, ibid.).

A study by Donaghy (Growth hormone, insulinlike growth factor-1, and insulinlike growth factor binding proteins 1 and 3 in chronic liver disease: Hepatology. 1995 March; 21(3):680-8) showed that in chronic liver disease there are significant changes in the levels of two of the major IGF-1 binding proteins, IGF-1 binding protein 1 (IGFBP-1) and IGF-1 binding protein 3 (IGFBP-3), respectively, and the study concluded that these changes may further limit the bioavailability of already low levels of circulating IGF-1. In particular the levels of IGFBP-3 were low in the individuals examined.

Low circulating levels of IGFBP-3 do not appear to be caused by a reduced gene transcription or an increased protease activity, and Ross (Expression of IGF-1 and IGF-binding protein genes in cirrhotic liver: J Endocrinol. 1996 May; 149(2):209-16) showed that hepatocytes remain transcriptionally active in cirrhosis, and that mechanisms apart from lack of expression may be responsible for the observed low levels of IGF-1 and IGFBP-3. Likely mechanisms could possibly affect translational alterations and/or modify the half-life of circulating IGF-1 and IGFBP-3.

Reduced levels of IGFBP-3 have not been observed in all cases. Picardi (ibid.) reported increased levels of IGFBP-3 in sera from cirrhotic rats and stated that according to current state of the art thinking, increased IGFBP-3 may reduce the tissue availability of IGF-1 by preventing interaction between the hormone and its tissue receptor.

In contrast to the low levels of IGFBP-3 reported by Donaghy (ibid.), a study by Kratzsch (Regulation of growth hormone (GH), insulin-like growth factor (IGF)I, IGF binding proteins −1, −2, −3 and GH binding protein during progression of liver cirrhosis: Exp Clin Endocrinol Diabetes. 1995; 103(5):285-91) showed that IGFBP-1 and IGFBP-2 levels were significantly elevated in preterminal liver disease suggesting an upregulatory mechanism is still effective in this situation. Only when liver function had markedly deteriorated, the serum levels of these two parameters decreased again, possibly due to an impaired synthesis.

### SUMMARY OF THE INVENTION

The present invention relates to IGF-1 treatment of an individual, such as e.g. a human being, suffering from an acute or chronic liver disease including hepatic cirrhosis. The efficacy of the present invention is demonstrated by a series of clinical trials performed with administration of IGF-1 to patients suffering from acute or chronic liver disease including cirrhosis. Recombinant human IGF-1 (rh IGF-1) has never before been tested in a human being suffering from liver cirrhosis.

Acute and chronic liver disease according to the invention are characterized by low circulating IGF-1 and IGFBP3 levels. As IGF-1 is reported to be an important anabolic hormone, this may contribute to the compromised carbohydrate metabolism of liver cirrhotic patients and the deranged amino acid metabolism. These metabolic disturbances leads to malnutrition which—according to the present invention—represents one important independent variable for the survival of patients with liver cirrhosis.

Circulating IGF-1 levels are only about one-half to one-third of the normal levels in patients with clinically evident cirrhosis of all types, and the concentration decreases progressively with worsening of Child-Pugh score (Comfriez 1991, Cuneo 1995, Assy 1997, Scheuf 1996, Donaghy 1997). Growth hormone levels, by contrast, increase progressively in patients with more advanced disease and are increased up to several fold, in Child-Pugh class C patients (Scheuf 1997), and the IGF-1 response to exogenous GH are nearly absent. Doses of GH which produce a near doubling of total IGF-1 levels in normal subjects produce only about 10% increase in Child-Pugh C class patients (Assy 1997).

The decline in circulating total IGF-1 concentration may well reflect a loss of liver function, and IGF-1 concentrations have been shown to correlate with serum albumin concentrations and to be an independent predictor of survival in a cohort of 354 cirrhotics followed prospectively (Møller 1996). Although IGFBP-3 levels are also decreased in patients with cirrhosis, the concentration of free IGF-1 is decreased in rough proportion to total IGF-1 levels (Assy 1997). Cirrhotic patients have therefore low circulating concentrations of total and free IGF-1 as well as low IGFBP-3 concentrations.

Collectively, these findings suggest that the progressively decline in total circulating IGF-1 levels and IGF-1 bioactivity (i.e.free IGF-1) in cirrhosis reflect a progressive decline in liver function, and IGF-1 levels, as well as the IGF-1 response to GH, have been proposed for assessing liver function and prognosis (Assy 1997, Møller 1996).

Patients with cirrhosis and malnutrion exhibit certain metabolic features normally characteristic of prolonged fasting. Endogenous glucose production due to hepatic gluconeogenesis is dramatically increased in both rats rats with experimental cirrhosis and in human cirrhotics, and proteolysis of muscle is increased, helping to fuel hepatic gluconeogenesis. This contributes to the hyperaminoacidemia often seen in these patients. (Petersen 1997, Petersen 1998). Unlike the normal situation, however, serum levels of insulin and glucose tend to be high rather than low (Shnueli 1992, Peterson 1997), findings characterisitic of insulin resistance.

Cirrhosis is characterized by a significant reduction in peripheral glucose disposal into muscle, in addition to failure of the normal insulin-mediated suppression of muscle proteolysis and hepatic gluconeogenesis (Peterson 1997). The basis for the observed insulin resistance is unknown, but possibilities include diminished level of IGF-1, high levels of glucagons or cathecolamines (Petersen 1997). Glucose clamp studies suggest that neither glucagons nor GH can account for the the insulin resistance in cirrhosis (Petersen 1997, Shmueli 1996).

IGF-1 deficiency is one plausible contributing factor to the catabolic state seen in cirrhosis. According to one proposed hypothesis according to the present invention, the anabolic effects of IGF-1 with respect to i) carbohydrate metabolism (promotes glucose uptake and glycogen formation by muscle, inhibits hepatic gluconeogenesis), and ii) protein metabolism (inhibits proteolysis, promotes amino acid synthesis and net protein build up in muscle) will benefit patients with cirrhosis. Additionally, IGF-1 therapy might well be expected to result in a decrease of GH levels, which might in turn decrease GH induced insulin resistance.

Although it has long been recognised that the growth hormone(GH)-IGF-1 axis is deranged in cirrhosis, the clinical trials disclosed herein represent the first systematic study of administration of recombinant, human IGF-1 to patients with chronic liver disease including cirrhosis. Patients with liver cirrhosis were treated with IGF-1 for one week and found marked improvement in both carbohydrate and amino acid metabolism. By combining IGF-1 with IGFBP3 it is hypothesised that the the biological effect of IGF-1 will be enhanced as IGF-1 on its own has a short half-life in circulation.

According to one preferred embodiment of the present invention, IGF-1 is administrered to a human being subcutaneously, preferably in the thigh or the abdominal skin, and preferably in two daily doses of about 50 microgram/kg twice a day. The present invention demonstrates that this dosis regime is able to restore normal IGF-1 levels in patients with liver cirrhosis, and the dose is well-tolerated by the patients.

In other aspects the invention is directed to treatment of an individual, preferably a human being, suffering from i) a liver disease occurring as a consequence of diabetes mellitus, and/or ii) diabetes mellitus and abnormalities of glucose homeostasis occurring as a complication of liver disease, and/or iii) liver disease occurring coincidentally with diabetes mellitus and/or abnormalities of glucose homeostasis.

The importance of the present invention is illustrated by the fact that the prevalence of type 1 diabetes in the United States is ˜0.26%. The prevalence of type 2 diabetes is far higher, ˜1-2% in Caucasian Americans and up to 40% in Pima Indians. According to the Centers for Disease Control and Prevention, hepatitis C alone chronically infects more than 1.8% of the American population, or more than 4 million people. It would not be unusual for these two diseases to occur by chance in the same person, which explains in part a possible association between the occurence of liver disease and diabetes mellitus.

In order to fully understand the scope of the present invention, one must first contemplate the complexities of liver metabolism. The liver plays a central and crucial role in the regulation of carbohydrate metabolism. Its normal functioning is essential for the maintenance of blood glucose levels and of a continued supply to organs that require a glucose energy source. This central role for the liver in glucose homeostasis offers a clue to the pathogenesis of glucose intolerance in liver diseases, but little insight into the mechanisms of liver disease in diabetes mellitus.

The Role of the Liver in Glucose Homeostasis

An appreciation of the role of the liver in the regulation of carbohydrate homeostasis is essential to understanding the many physical and biochemical alterations that occur in the liver in the presence of diabetes and to understanding how liver disease may affect glucose metabolism. It will then be clear how treatment of a liver disease according to the present invention may also affect diseases associated with glucose metabolism. The diseases and indications described herein below are meant as an illustration of the conditions and illnesses in an individual, such as e.g. a human being, that may be subjected to treatment according to the present invention.

The liver uses glucose as a fuel and also has the ability to store it as glycogen and synthesize it from noncarbohydrate precursors (gluconeogenesis). Mann and Magath demonstrated that a total hepatectomy in a dog results in death within a few hours from hypoglycemic shock,^{1,2} underscoring the important role the liver plays in maintaining normoglycemia.

Glucose absorbed from the intestinal tract is transported via the portal vein to the liver. Although the absolute fate of this glucose is still controversial, some authors suggest that most of the absorbed glucose is retained by the liver so that the rise in peripheral glucose concentration reflects only a minor component of postprandial absorbed glucose. Therefore, it is possible that the liver plays a more significant role than does peripheral tissue in the regulation of systemic blood glucose levels following a meal.³ Katz and associates,⁴ however, suggest that most absorbed glucose is not taken up by the liver but is rather metabolized via glycolysis in the peripheral tissues.

Many cells in the body, including fat, liver, and muscle cells, have specific cell membrane insulin receptors, and insulin facilitates the uptake and utilization of glucose by these cells. Glucose rapidly equilibrates between the liver cytosol and the extracellular fluid. Transport into certain cells, such as resting muscle, is tightly regulated by insulin, whereas uptake into the nervous system is not insulin-dependent.

Glucose can be used as a fuel or stored in a macromolecular form as polymers: starch in plants and glycogen in animals. Glycogen storage is promoted by insulin, but the capacity within tissues is physically limited because it is a bulky molecule. Insulin is formed from a precursor molecule, preproinsulin, which is then cleaved to proinsulin. Further maturation results in the conversion of proinsulin into insulin and a smaller peptide called C-peptide. A small amount of proinsulin enters the circulation. It has a half-life 3-4 times longer than that of insulin because it is not metabolized by the liver. However, proinsulin has <10% of the biological activity of insulin.

Insulin is metabolized by insulinase in the liver, kidney, and placenta. About 50% of insulin secreted by the pancreas is removed by first-pass extraction in the liver. Insulin promotes glycogen synthesis (glycogenesis) in the liver and inhibits its breakdown (glycogenolysis). It promotes protein, cholesterol, and triglyceride synthesis and stimulates formation of very-low-density lipoprotein cholesterol. It also inhibits hepatic gluconeogenesis, stimulates glycolysis, and inhibits ketogenesis. The liver is the primary target organ for glucagon action, where it promotes glycogenolysis, gluconeogenesis, and ketogenesis.^{5,6}

Glucose that is taken up by a cell may be oxidized to form energy (glycolysis). It is oxidized to pyruvate in the cytosol, and electrons generated from this process are transferred to the mitochondria. Pyruvate generated by this Emden-Meyerhof pathway is oxidized to acetyl CoA in the mitochondria, which in turn undergoes further oxidation by the Krebs tricarboxylic acid cycle. Nearly 36 moles of high energy phosphate are generated from each molecule of glucose by aerobic glycolysis. Should oxygen not be available, pyruvate is converted to lactate by the action of lactate dehydrogenase. Lactate is a potential fuel, or it may be converted back to glucose. The formation of glucose from lactate and various noncarbohydrate precursors is known as gluconeogenesis and occurs mainly in the liver and kidneys.

The liver, kidney, intestine, and platelets contain the enzyme glucose-6-phosphatase, which produces glucose from glucose-6-phosphate and is the final step in the production of glucose via gluconeogenesis. This enzyme is absent in other tissues. Glucose that is metabolized peripherally may therefore be converted back to glucose or to hepatic glycogen via gluconeogenesis with lactate as the primary substrate.⁷ This is known as the Cori cycle.

In type 2 diabetes, excessive hepatic glucose output contributes to the fasting hyperglycemia. Increased gluconeogenesis is the predominant mechanism responsible for this increased glucose output, while glycogenolysis has not been shown to be increased in patients with type 2 diabetes.⁸ Hyperglucagonemia has been shown to augment increased rates of hepatic glucose output, probably through enhanced gluconeogenesis.

Liver Disease Occurring as a Consequence of Diabetes Mellitus

Glycogen Deposition

Excess glycogen accumulation in the liver is seen in 80% of diabetic patients.⁹ Glycogen synthesis in the liver is impaired in diabetes due to defective activation of glycogen synthase. However, studies attesting to this were usually performed on animals with recently induced diabetes. In patients with chronic diabetes, glycogen accumulation is seen, and it is postulated that long-standing insulin deficiency may actually facilitate synthase activity. This and enhanced gluconeogenesis may account for the net accumulation of glycogen in diabetes.¹⁰

The mechanism of cytoplasmic glycogen deposition is uncertain but is perhaps related to the large variations in glucose concentration and frequent insulin dosing. No correlation between hepatic glycogen content and fasting blood glucose levels has been demonstrated. There is also no demonstrable association between the type of diabetes or the fat content of the hepatocytes and the presence of glycogen. The mechanism for nuclear glycogen deposition is also unclear, with the stored glycogen resembling muscle glycogen more than hepatocyte cytoplasmic glycogen.¹¹⁻¹³ Nuclear glycogen deposition was first described by Ehrlich in 1883.¹⁴ It is postulated that glycogen is actually synthesized in the nucleus and has been found in 60-75% of diabetic patients.^{15,16} Nuclear glycogen deposition is also seen in sepsis, tuberculosis, some patients with hepatitis (particularly autoimmune chronic hepatitis), Wilson's disease, and cirrhosis. The finding of glycogen nuclei in a patient with fatty liver is useful confirmatory evidence that the fatty liver is secondary to diabetes even if the glucose tolerance test is normal. However, Creutzfeldt and associates have reported the combination also in obese patients.¹⁷⁻¹⁹

Patients showing solely excessive glycogen deposition may exhibit hepatomegaly and liver enzyme abnormalities and may have abdominal pain and even nausea and vomiting and rarely ascites. All these abnormalities may improve with sustained glucose control.²⁰

Fatty Liver, Steatohepatitis

Hepatic fat accumulation is a well-recognized complication of diabetes with a reported frequency of 40-70%. Unfortunately, associated obesity is a frequently occurring confounding variable. Type 1 diabetes is not associated with fat accumulation if glycemia is well controlled, but type 2 diabetes may have a 70% correlation regardless of blood glucose control.

Fat is stored in the form of triglyceride and may be a manifestation of increased fat transport to the liver, enhanced hepatic fat synthesis, and decreased oxidation or removal of fat from the liver. The steatosis may be microvesicular or macrovesicular and may progress to fibrosis and cirrhosis. The degree of glycemic control does not correlate with the presence or absence of fat.²¹⁻²⁶ The most common clinical presentation is hepatomegaly, and most patients have normal or only mildly abnormal transaminases and normal bilirubin.

CT scan and ultrasound are claimed to be sensitive tests for detecting hepatic fat accumulation. A negative ultrasound, however, does not exclude the presence of microscopic fatty infiltration.²⁷ A liver biopsy is obviously the best method for detecting hepatic fat accumulation. It is unclear at this time whether a biopsy is always necessary in patients with suspected steatohepatitis. Biopsy probably should be performed when the diagnosis is unclear, although some authors suggest that it is necessary in all cases to confirm the diagnosis and assess the degree of fibro.^{28,29}

Excessive fat accumulation is seen in alcoholic liver disease, obesity, prolonged parenteral nutrition, protein malnutrition, jejunoileal bypass, and chronic illnesses complicated by impaired nutrition, such as ulcerative colitis and chronic pancreatitis. It can also occur as a result of hepatotoxins, such as carbon tetrachloride, and can be seen in association with abetalipoproteinemia, Weber-Christian disease, the HIV virus, cholesterol ester storage disease, and Wilson's disease, in addition to diabetes mellitus. A number of drugs, such as amiodarone, perhexilene, glucocorticoids, estrogens, and tamoxifen, may cause macrovesicular steatosis. The amount of fat frequently diminishes with improvement of the underlying condition.

Nonalcoholic steatohepatitis (NASH) is a variant of fatty liver in which fat in the hepatocytes is accompanied by lobular inflammation and steatonecrosis. The diagnosis can only be made in the absence of alcohol abuse or other causes of liver disease, particularly hepatitis C. In patients with diabetes and steatohepatitis, Mallory bodies such as those seen in alcoholic liver disease may be seen. Nonalcoholic steatohepatitis has been associated most commonly with obese women with diabetes, but the disease is certainly not limited to patients with this clinical profile.³⁰ There is certainly a higher prevalence in type 2 diabetic patients on insulin.³¹

The spectrum of clinical disease in fatty liver with steatohepatitis varies from the asymptomatic elevation of liver enzymes to severe liver disease with fibrosis and nodular regeneration. Patients with nonalcoholic steatohepatitis can develop progressive liver disease and complications to the point that they may need liver transplantation.³²

Nonalcoholic steatohepatitis should be considered as a cause for chronically elevated liver enzymes in asymptomatic diabetic patients particularly if they are obese and have hyperlipidemia.³³ In type 2 diabetic patients with or without obesity, up to 30% have fat with inflammation, 25% have associated fibrosis, and 1-8% have cirrhosis.³⁴⁻³⁶

The morphological pattern of diabetic steatohepatitis resembles that seen in alcoholic hepatitis. However, the histopathological changes in diabetes tend to be periportal (situated in zone I), while those in alcoholic hepatitis are predominantly pericentral (in zone III). It is not clear whether the diabetes is causally related to the steatohepatitis.^{37,38} In an animal model of type 1 diabetes, there is a high incidence of perisinusoidal hepatic fibrosis, while in humans perisinusoidal fibrosis often parallels with diabetic microangiopathy.³⁹

Gradual weight loss and good control of blood glucose levels is recommended for patients with steatohepatitis, since rapid weight loss may actually worsen NASH.^{40,41} Weight loss >10% has been shown to be necessary for normalization of liver enzymes in patients who are significantly overweight.⁴² Ursodeoxycholic acid may be beneficial in reducing steatosis and may result in normalization of liver enzymes and improvement in histology without demonstrable impact on fibrosis.⁴³⁻⁴⁵

Cirrhosis

There is an increased incidence of cirrhosis in diabetic patients, and, conversely, at least 80% of patients with cirrhosis have glucose intolerance.^{46,47} The reported prevalence of cirrhosis in diabetes varies widely. Diabetes increases the risk of steatohepatitis, which can progress to cirrhosis. Obesity is a significant confounding variable in determining the prevalence of cirrhosis in diabetes. Even with normal glucose tolerance, obesity can cause steatohepatitis and cirrhosis. Likewise, the lack of a clear definition of diabetes in the past somewhat confounds these statistics.

Biliary Disease, Cholelithiasis, Cholecystitis

There is a reported increased incidence of cholelithiasis in diabetes mellitus, but obesity and hyperlipidemia may again be confounding variables. Several articles have reported a two- to threefold increased incidence of gallstones in diabetic patients, whereas others have failed to demonstrate a significant association.^{17,48-52} Gallbladder emptying abnormalities found in diabetic patients may predispose patients to cholelithiasis.⁵³ Secretion of lithogenic bile by the liver in patients with type 2 diabetes probably predisposes them to forming gallstones, but this is likely a result of concomitant obesity rather than a result of the diabetes itself.⁵⁴ Increased biliary cholesterol saturation has not been demonstrated in insulin-dependent diabetic patients.

There is no indication in the literature that the natural history of gallstones is different in diabetic and nondiabetic individuals. The relative risk of mortality following acute cholecystitis is not significantly greater in diabetic patients than in the general population, and neither is the risk for serious complications. For that reason, prophylactic cholecystectomy cannot routinely be recommended for asymptomatic gallstones in patients with diabetes.⁵⁵ Any increase in mortality may be attributed to underlying renal or vascular disease. Patients with diabetes have comparable survival outcomes from laparoscopic or open cholecystectomy.⁵⁶

Complications of Diabetes Therapy

Insulin therapy may increase the risk of a patient of acquiring viral hepatitis because of the exposure to needles. Adhering to good infection-control practices should significantly reduce this risk.

The biguanide metformin (Glucophage) does not undergo hepatic metabolism and, like chlorpropamide (Diabinese), is excreted unchanged in the urine.⁵⁷ In contrast, the sulfonylurea glyburide (Micronase, Glynase, Diabeta) is excreted in bile and urine in a 50/50 ratio. The sulfonylurea glipizide (Glucotrol, Glucotrol XL) is metabolized mainly by the liver, and, in theory, hepatic disease may result in increased blood levels.

There is a rare association between the use of oral hypoglycemics and hepatic injury, but sulfonylureas can cause chronic hepatitis with necroinflammatory changes.⁵⁸ Granulomatous changes can also be seen. They are described as having a well-circumscribed cellular infiltrate comprised of acidophilic histiocytes and eosinophils surrounding necrotic hepatocytes. The mechanism of liver injury is not known.

Chlorpropamide appears to be the most hepatotoxic of these drugs, with cholestatic hepatitis occurring in 0.5% of people on the drug. Jaundice develops over 2-5 weeks and resolves in virtually all patients when the drug is stopped. Hepatic disease is very rare with tolbutamide (Orinase and generics), and tolazamide (Tolinase and generics). Although very uncommon, acetohexamide and glyburide can cause acute hepatocellular necrosis, and fatalities have been reported. At least two cases of granulomatous hepatitis thought secondary to glyburide have been reported in the literature.⁵⁹

The biguanides, such as metformin hydrochloride, have not been associated with liver injury. Lactic acidosis can be associated with the use of metformin to treat diabetes, but it is reported to occur occasionally and usually in patients with major contraindications to the drug. “Chronic liver disease” is one of the conditions that may predispose patients taking metformin to developing lactic acidosis, probably due to a reduced ability of the liver to clear lactate. It is therefore listed as a contraindication.⁶⁰

Troglitazone (Rezulin) is an oral antihyperglycemic agent that acts primarily by decreasing insulin resistance. Its package insert carries a warning that severe idiosyncratic hepatocellular injury, usually reversible but possibly leading to death or liver transplantation, has been reported in patients using the medication, usually during the early months of therapy.

Serum transaminases should be checked at the start of therapy, monthly for the first 6 months of therapy, every 2 months for the remainder of the first year, and periodically thereafter. If a patient's ALT level is >3 times the upper limit of normal, therapy should not be started or should be discontinued in those already receiving the medication. In patients with levels >1.5 times the upper limit of normal, repeat evaluations at earlier intervals are necessary to ensure that more serious deterioration of liver enzymes is not developing. In addition, any symptoms suggesting hepatic dysfunction necessitate having liver tests performed.

Diabetes and Abnormalities of Glucose Homeostasis Occurring as a Complication of Liver Disease

Viral Hepatitis

There is no evidence in the literature that viral hepatitis has a worse prognosis in patients with diabetes. There is an increased prevalence of viral hepatitis in diabetes possibly due to an increased exposure to needles for the injection of insulin or for blood testing. Possible contamination of the platform in spring-loaded lancet devices may increase the risk of acquiring hepatitis B or C from these instruments. In 1996, hepatitis B outbreaks were noted in an Ohio nursing home and a New York hospital. Transmission was thought to be related to the use of spring-loaded devices for fingerstick glucose testing.^{61,62}

Diabetes is far more prevalent in patients with hepatitis C than in patients with other forms of viral hepatitis. In a study by Grimbert and associates, 152 patients with hepatitis C and the same number with either hepatitis B or alcohol-induced liver disease were compared over the same period. Twenty-four percent of the patients with hepatitis C had diabetes compared with only 9% of the controls. The authors suggested a causative role of hepatitis C in the pathogenesis of diabetes.⁶³

Fraser and associates also found an association between chronic hepatitis C and the presence of impaired glucose control and reported that the prevalence of diabetes was much higher in hepatitis C than in the general population.⁶⁴ One hundred adults with cirrhosis were evaluated in a retrospective study. Of the 34 patients with hepatitis C, 50% had diabetes mellitus, as opposed to 9% of the 66 patients with cirrhosis unrelated to hepatitis C. The association has been described also by others and was thought to be statistically significant.^{65,67}

Simo and associates also suggested that the hepatitis C virus may have a direct causative role in the development of diabetes. Most of their diabetic patients with hepatitis C had abnormal liver tests.⁶⁸

The association of diabetes with hepatitis C has also been investigated in posttransplantation patients, and there is a reported higher incidence of diabetes in liver transplant recipients with hepatitis C. This increased incidence appears to be significant, and the presence of the virus appears to be an independent risk factor.⁶⁹ Interferon therapy used to treat hepatitis B and C may induce hyperglycemia, result in the development of type 2 diabetes, and necessitate increased insulin requirements in patients with type 1 diabetes.⁷⁰⁻⁷³ Interferon therapy has resulted in the development of type 1 diabetes likely through the development of insulin autoantibodies.⁷⁴⁻⁷⁶ Faftovich and associates retrospectively studied 11,241 patients with chronic viral hepatitis who had undergone interferon therapy. However, only 10 patients developed de novo diabetes mellitus.⁷⁷ Interferon therapy also reportedly led to severe hypertriglyceridemia in a diabetic patient.⁷⁸

The hepatitis B vaccine effectively induces protective antibodies in most patients with diabetes.^{79,80} One study in children with type 1 diabetes concluded that children may not respond as well to the vaccination. This suggested that children should perhaps be vaccinated with four injections instead of three.⁸¹

Cirrhosis

Individuals with cirrhosis have elevated insulin levels, perhaps indicating insulin resistance or reduced degradation of insulin by the cirrhotic liver. In the absence of peripheral insulin resistance, it is likely that patients with cirrhosis would become hypoglycemic.

The pathogenesis of the proposed insulin resistance is not known, although a receptor or postreceptor abnormality is postulated.⁸² Impaired insulin secretion from the pancreatic-cells has been proposed as another cause for the hyperglycemia,⁸³ and glucose intolerance in patients with decompensated cirrhosis has been found to be associated with low insulin secretion.⁸⁴ Potassium depletion, excess glucagon, growth hormone, cortisol, and increased fatty acid levels in blood, and reduced insulin receptors may account for the insulin resistance, but these are all unproved hypotheses.

Cirrhotic patients may develop fasting hypoglycemia by way of the “Insulin Autoimmune Syndrome” associated with the development of high levels of insulin autoantibodies even in the absence of hepatocellular carcinoma.⁸⁵ Cirrhotic patients and patients with fulminant hepatic failure may have lower blood glucose concentrations than matched subjects, but significant hypoglycemia may be prevented by decreased utilization of glucose and an increased utilization of nonglucose fuels such as fat.₈₆₋₈₈

Cirrhosis according to the present invention comprises all classes of cirrhosis, such as for example Cirrhosis Child's class A, B, C or D, wherein A designates the mildest and D the most severe class of cirrhosis.

Hepatocellular Carcinoma

Hepatocellular carcinoma may be associated with the development of hypoglycemia. A proposed mechanism for the development of this hypoglycemia is the production of insulin-like growth factor-II (IGF-II) by hepatocellular carcinoma cells (HCC). Numerous case reports have discussed the development of this phenomenon.

IGF-1 I is a protein that functions as a partial insulin agonist.⁸⁹ Diabetic patients who develop HCC may require progressively less insulin, not only due to the production of IGFs, but also due to increased glucose utilization by insulin-sensitive tissue.⁹⁰⁻⁹³ A study by Adami and associates on a cohort of about 154,000 patients suggested that patients with diabetes are at increased risk for developing primary liver cancer.⁹⁴ In a case-controlled study in Italy, it was again suggested that patients with diabetes may be at higher risk for hepatocellular carcinoma, although the reason why is unclear.⁹⁵

Fulminant Hepatic Failure

Fulminant hepatic failure may be complicated by hypoglycemia, and its development may portend a poor prognosis and increased mortality.^{96,97} Such patients need to be closely observed, and most require glucose supplementation. Destruction of hepatocytes along with hyperinsulinism and inadequate storage of glucose in extrahepatic organs contributes to the hypoglycemia.⁹⁸

Liver Transplantation and Diabetes

The issue has been raised whether the presence of diabetes before or after liver transplantation influences the outcome. Carson and Hunt reported a 4-20% incidence of posttransplant diabetes following liver transplantation.⁹⁹ Trail and associates retrospectively investigated 497 patients who had received orthotopic liver transplants. Twenty-six patients (5.2%) had clinical evidence of diabetes 1 month after transplant. This did not influence graft survival, liver synthetic function, or number of rejection episodes during the first year. The investigators concluded that the presence of posttransplant diabetes did not significantly affect patient outcome in the first year.¹⁰⁰

Navasa and associates evaluated 102 patients who survived longer than 1 year after orthotopic liver transplantation. Fourteen had diabetes before transplantation, and all but one were alive 3 years later. Their reported incidence of posttransplant diabetes was 27% at 1 year, 9% at 2 years, and 7% at 3 years. Patients with post-transplant diabetes had a significantly higher mortality in the second postoperative year than did patients without this complication. This may be related to an increased use of immunosuppressive agents in those patients with rejection and thus a greater predisposition to diabetes.¹⁰¹

Fk506, tacrolimus (Prograf), a potent immunosuppressive agent used in liver transplantation to prevent allograft rejection, may cause diabetes mellitus. Stopping the drug may result in restoration of normal glucose tolerance.¹⁰² Liver transplantation may be performed in patients with type 1 diabetes without any increased risk for graft or patient survival regardless of the underlying liver disease indication. Interestingly, in patients with renal transplants, both diabetes and hepatitis B were associated with less favorable outcomes.¹⁰³

Liver Disease Coincident with Diabetes and Abnormalities of Glucose Homeostasis

Hemochromatosis

Hemochromatosis is an autosomal recessive inherited condition characterized by an abnormally high absorption of iron from the small intestine and excessive accumulation of iron in the liver and other tissues. Most patients (>80%) with the hemochromatosis (HFE) gene have one of the two described gene mutations, namely, the Cys282Tyr mutation, situated on the short arm of chromosome six. Patients with untreated hemochromatosis develop progressive liver disease, cirrhosis, and diabetes and are at high risk for developing hepatocellular carcinoma.¹⁰⁴ The term “bronze diabetes,” coined by Hanot and Schachmann in 1886, refers to the association of diabetes with hemochromatosis.⁹ About 75% of patients with hemochromatosis and established cirrhosis have diabetes. Patients with hemochromatosis and diabetes have a significantly reduced survival compared to hemochromatosis patients without diabetes.¹⁰⁵

Hemochromatosis is the most common single gene-inherited metabolic disease amongst Caucasians worldwide. The heterozygote frequency is about 10%; one in 250 people are homozygotes. Patients with hemochromatosis and diabetes have both impaired insulin secretion and increased insulin resistance. ¹⁰⁶ The likelihood of diabetes in patients with hemochromatosis increases as the liver iron concentration increases.¹⁰⁷

Whether all diabetic patients should be screened for hemochromatosis has been considered. Turnbull and associates evaluated 727 patients in a diabetic clinic. Of those, 7.4% had elevated iron indices on initial screening, and in 3% these indices remained elevated on fasting blood specimens. However, only one had homozygous hereditary hemochromatosis, leading to their conclusion that routine screening for hemochromatosis in diabetic patients is probably not cost-effective.¹⁰⁸ In contrast, patients with diabetes who have a family history of liver disease should probably be screened for hemochromatosis.

Excessive iron accumulation in conditions other than hemochromatosis, such as dyserythropoietic disorders, may also be associated with diabetes. The pancreatic-cell may recover to varying degrees when the excess iron is removed in conditions associated with iron overload,^{109,110} but rarely will phlebotomy therapy restore normal glucose tolerance.¹⁰⁵

Glycogen Storage Disease

Absence of glucose-6-phosphatase or other enzymes necessary for glycogen degradation, as occurs in a variety of glycogen storage diseases, would prevent the use of stored glycogen to maintain the blood glucose concentration in the fasting state. An infant so affected may require carbohydrate feedings every 2-3 hours to prevent possible brain damage. Glycogen content in the livers of most of these affected patients is excessive. The most common form is type 1 glycogenesis, characterized by a deficiency of the enzyme glucose-6-phosphatase. It is inherited in an autosomal recessive fashion.¹¹¹

Autoimmune Biliary Disease

Type 1 diabetes may be one of the manifestations of the autoimmune polyglandular syndrome. Primary biliary cirrhosis (PBC) has been reported in a patient with this syndrome, raising the possibility that PBC may be an associated autoimmune manifestation of this condition.¹¹²

Primary sclerosing cholangitis (PSC), which involves to varying degrees the intrahepatic and extrahepatic biliary tree and which may progress to cirrhosis, can also involve the pancreatic duct and result in chronic inflammatory pancreatic changes. The pancreatic changes may be severe enough to cause functional changes and may result in glucose intolerance.¹¹³

It is also postulated that ulcerative colitis, sclerosing cholangitis, and diabetes may occur in the same patient as part of a generalized genetically determined autoimmune disease influenced by HLA genotype. Glucose intolerance may be higher in patients with PSC than in patients with other liver disease.¹¹⁴⁻¹¹⁶

In conclusion, the association between diabetes and liver disease has relevance to diabetologists, hepatologists, and primary care physicians. The finding of an excess prevalence of chronic liver disease in type 2 diabetic patients has stimulated interest in this association and on exploration of avenues of pathogenesis that promise to shed light on the relationship between hepatic metabolism and glucose homeostasis. This review attempted to summarize some of these associations. While it raises more questions than it answers, hopefully future research will fill in the gaps in our current understanding of this intriguing link between two major disease entities.

In one preferred embodiment the present invention pertains to the treatment of liver disease in an individual, preferably a human being, suffering from diabetes mellitus.

In another embodiments the invention is directed to treatment of an individual, preferably a human being, suffering from a liver disease occurring as a consequence of diabetes mellitus.

In yet another embodiment there is provided a method for treating an individual, preferably a human being, suffering from diabetes mellitus and abnormalities of glucose homeostasis occurring as a complication of liver disease.

In a still further embodiment there is provided a method of treatment of an individual, preferably a human being, suffering from a liver disease occurring coincidentally with diabetes mellitus and/or abnormalities of glucose homeostasis.

When the present invention relates to a method of treatment of an individual, preferably a human being, suffering from a liver disease occurring as a consequence of diabetes mellitus such method involves, but is not limited to, treatment of any one or more of i) glycogen deposition, ii) steatosis and nonalcoholic steatohepatitis (NASH), iii) fibrosis and/or cirrhosis, iv) biliary disease, v) cholelithiasis, vi) cholecystitis, and vii) any complication of a method of therapy of diabetes including cholestatic complications and necroinflammatory complications, when such a condition occurs in said individual.

When the present invention relates to a method for treatment of an individual, preferably a human being, suffering from diabetes mellitus as well as from abnormalities of glucose homeostasis occurring as a complication of liver disease, such a method involves, but is not limited to, treatment of diabetes mellitus occuring as a complication of any liver disease including, but not limited to i) hepatitis, ii) cirrhosis, iii) hepatocellular carcinoma, iv) fulminant hepatic failure, and v) postorthotopic liver transplantation.

When the present invention is directed to a method for treatment of an individual, preferably a human being, suffering from a liver disease occurring coincidentally with diabetes mellitus and abnormalities of glucose homeostasis including, such a method involves, but is not limited to, treatment of liver disease in combination with any one or more of i) hemochromatosis, ii) glycogen storage disease, and iii) autoimmunebiliary disease.

Technical Terms and Definitions

Acid labile subunit (ALS): As used herein, “ALS” refers to the acid-labile, 84-86 kD, non-IGF-binding subunit of the 125-150 kD complex With IGFBP-3 and IGF-1 as described in Baxter, WO 90/0569, or any animal equivalent thereof, preferably human ALS. It may be from any source, including natural, synthetic, or recombinant sources.

Acute liver disease: Any desease of the liver rapidly developing into to a critical stage. Acute liver disease is normally non-persistent as compared to chronic liver disease. The disease is diagnosed as any alteration in the state of the liver interrupting or disturbing the performance of vital liver functions, and causing or threatening any one or more of pain, weakness, malady, illness, sickness, and disorder.

Ameliorating method of treatment: Any treatment resulting in reversing at least partly any alteration in the state of the liver interrupting or disturbing the performance of vital liver functions, and reducing any one or more of pain, weakness, malady, illness, sickness, and disorder caused by said interruption or disturbance.

Ascites: Accumulation of fluid in the peritoneal cavity causing swelling. Frequent causes of ascites include infection of the liver and portal hypertension.

Cirrhosis of the liver: Substantially irreversible condition affecting the whole liver involving loss of parenchymal cells, inflammation, disruption of the normal tissue architecture, and eventually hepatic failure

Chronic hepatitis: Persistent, long-lasting hepatitis infection.

Chronic liver disease: Persistent, long-lasting desease of the liver substantially without any development, or slowly progressing into a critical stage. Chronic liver disease is normally persistent as compared to non-persistent, acute liver disease. The disease is diagnosed as any alteration in the state of the liver interrupting or disturbing the performance of vital liver functions, and causing or threatening any one or more of pain, weakness, malady, illness, sickness, and disorder.

Conservative amino acid substitution: Substitution of one amino acid (within a predetermined group of amino acids) for another amino acid (within the same group) exhibiting similar or substantially similar characteristics. Within the meaning of the term “conservative amino acid substitution” as applied herein, one amino acid may be substituted for another within groups of amino acids characterised by having

i) polar side chains (Asp, Glu, Lys, Arg, His, Asn, Gin, Ser, Thr, Tyr, and Cys,)

ii) non-polar side chains (Gly, Ala, Val, Leu, Ile, Phe, Trp, Pro, and Met)

iii) aliphatic side chains (Gly, Ala Val, Leu, Ile)

iv) cyclic side chains (Phe, Tyr, Trp, His, Pro)

v) aromatic side chains (Phe, Tyr, Trp)

vi) acidic side chains (Asp, Glu)

vii) basic side chains (Lys, Arg, His)

viii) amide side chains (Asn, Gin)

ix) hydroxy side chains (Ser, Thr)

x) sulphor-containing side chains (Cys, Met), and

xi) amino acids being monoamino-dicarboxylic acids or monoaminomonocarboxylic-monoamidocarboxylic acids (Asp, Glu, Asn, Gin).

Curative method of treatment: Any treatment resulting in substantially reversing any alteration in the state of the liver interrupting or disturbing the performance of vital liver functions and bringing the state of the liver back to a physiological state existing prior to the unset of said interruption or disturbance, and substantially reducing and/or eliminating any one or more of pain, weakness, malady, illness, sickness, and disorder caused by said interruption or disturbance.

Deficiency of IGF-1: Decreased levels of serum IGF-1

Diabetes mellitus: Condition of an individual characterised by a relative or absolute lack of insulin leading to uncontrolled carbohydrate metabolism.

Fibrosis of the liver: Condition characterised by deposition of an avascular collagenrich matrix in a wound, usually as a consequence of slow fibrinolysis or extensive tissue damage, as in sites of chronic inflammation of the liver.

Hepatic encephalopathy: Condition used to describe the deleterious effects of liver failure on the central nervous system. Features include confusion ranging to unresponsiveness (coma). A common cause is alcoholic cirrhosis.

Hepatic nephropathy: Condition involved in establishing hepatorenal syndrome.

IGF-1: Insulin-like growth factor 1 from any species, including bovine, ovine, porcine, equine, avian, and preferably human, in native-sequence or in variant form, and from any source, whether natural, synthetic, or recombinant.

Preferred herein for animal use is that form of IGF-1 from the particular species being treated, such as porcine IGF-1 to treat pigs, ovine IGF-1 to treat sheep, bovine IGF-1 to treat cattle, etc.

Preferred herein for human use is human native-sequence, mature IGF-1, more preferably without a N-terminal methionine, prepared, e.g., by the process described in EP 230,869 published Aug. 5, 1987; EP 128,733 published Dec. 19, 1984; or EP 288,451 published Oct. 26, 1988. More preferably, this native-sequence IGF-1 is recombinantly produced and is available from Genentech, Inc., South San Francisco, Calif., for clinical investigations. Also preferred for use is IGF-1 that has a specific activity greater than about 14,000 units/mg as determined by radioreceptor assay using placenta membranes, such as that available from KabiGen AB, Stockholm, Sweden.

IGF-1 binding protein: A protein capable of binding IGF-1 and modulating the biological action of IGF-1 at the cellular level. IGF-1 binding protein refers to any protein that binds IGF-1 in the circulation, in other body fluids, and in media conditioned by cultured cells, as defined in the Workshop on IGF Binding Proteins held in Vancouver, Canada in June 1989 discussed above and reported in Ballard et al., Acta Endocrinol. (Copenhagen), 121: 751-752 (1989). This term includes IGFBP-1, IGFBP-2, IGFBP-3, IGFBP-4, IGFBP-5, IGFBP-6, and other as yet unidentified IGFBPs that have the characteristics common to all the known IGF binding proteins. The term includes animal equivalents to human IGFBPs as well as human IGFBPs, for example, the bovine, ovine, porcine, and equine species. It may be from any source, whether natural, synthetic, or recombinant, provided that it will bind to the appropriate binding domain of IGF-1.

IGF-1 binding protein 3 (IGFBP-3): IGFBP-3 is defined as described above and in WO 89/09268 published Oct. 5, 1989 and Wood et al., Molecular Endocrinology, supra, but includes animal equivalents to human IGFBP-3 as well as human IGFBP-3, for example, the bovine, ovine, porcine, and equine species. It may be from any source, whether natural, synthetic, or recombinant, provided that it will bind to the appropriate binding domain of IGF-1.

Individual: Any mammalian species. Mammal signifies humans as well as animals. Mammals that are candidates for treatment include animals of economic importance such as bovine, ovine, and porcine animals. The preferred mammal herein is a human.

Insulin resistance: Condition wherein an individual diagnosed as having noninsulindependent diabetes produce insulin in sufficient amounts without being able to respond to the action of insulin.

Liver failure: A condition of severe end-stage liver dysfunction accompanied by a decline in mental status that may range from confusion (hepatic encephalopathy) to unresponsiveness (hepatic coma).

Liver functionality: Physiological condition established according to the present invention by ability of the liver to perform its normal functions with respect to glucose metabolism and amino acid metabolism. Normal glucose metabolism and normal amino acid metabolism signifies normal liver functionality.

Malnutrition: Any condition arising from intake of insufficient amounts of food, or intake of unballanced diet.

Metabolic disorder: Acquired condition caused by a failure of a metabolically important organ including the liver.

Pharmaceutically effective amount: Any amount of medicament capable of achieving a clinical effect in an individual treated with said medicament.

Portal hypertension: Hypertension of the portal chambers.

Prophylactic method of treatment: Method of treatment relating to prevention, or at least amelioration, of any disease including liver disease. Mammals “at risk” for ARF are those mammals, including mammals of economic importance such as bovine, ovine, and porcine animals, as well as humans, the latter being preferred, that are prone to exhibit ARF from operations or transplants to be performed or illnesses likely to be incurred.

Subcutaneous injection: Administration of a substance by means of an injection of the substance under the skin of an individual to which the substance is to be administered.

Variant: Variants of IGF-1 are described in relation to IGF-1 having a predetermined amino acid sequence such as e.g. the amino acid sequence of human IGF-1. Variants of IGF-1 fragments are described in relation to IGF-1 fragments having a predetermined amino acid sequence such as e.g. the amino acid sequence of a fragment of human IGF-1. Variants of IGF-1 binding proteins are described by similar analogy.

The following definitions shall denote an IGF-1 variant, or a fragment thereof, in relation to IGF-1, or a fragment thereof, having a predetermined amino acid sequence:

i) IGF-1 variants, or fragments thereof, comprising an amino acid sequence capable of being recognised by an antibody also capable of recognising IGF-1 having said predetermined amino acid sequence, and/or

ii) IGF-1 variants, or fragments thereof, comprising an amino acid sequence capable of binding to a receptor moiety, preferably an IGF-1 receptor moiety or an insulin receptor moiety, wherein said moiety is also capable of binding IGF-1 having said predetermined amino acid sequence, and/or

iii) IGF-1 variants, or fragments thereof, having at least a substantially similar anabolic action as compared to IGF-1 having said predetermined amino acid sequence.

Preferred IGF-1 variants are those e.g. described in U.S. Pat. No. 5,077,276 issued Dec. 31, 1991, in PCT WO 87/01038 published Feb. 26, 1987 and in PCT WO 89/05822 published Jun. 29, 1989, i.e., those wherein at least the glutamic acid residue is absent at position 3 from the N-terminus of the mature molecule, and those having a deletion of up to five amino acids at the N-terminus. The most preferred variant has the first three amino acids from the N-terminus deleted (variously designated as brain IGF, tlGF-1, des(1-3) IGF-1, or des-IGF-1).

Variants of IGF-1 binding proteins are polypeptides comprising an amino acid sequence capable of binding to IGF-1.

Preferred Embodiments of IGF-1 and Variants Thereof

IGF-1 as used herein relates to any insulin-like growth factor 1 from any species, including bovine, ovine, porcine, equine, avian, and preferably human, in native-sequence or in variant form, and from any source, whether natural, synthetic, or recombinant.

When an IGF-1 variant according to the invention is generated by a substitution of one amino acid for another, such a substitution may be a conservative amino acid substitution as defined herein. Fragments of IGF-1 according to the present invention may comprise more than one such substitution, such as e.g. two conservative amino acid substitutions, for example three or four conservative amino acid substtutions, such as five or six conservative amino acid substitutions, for example seven or eight conservative amino acid substitutions, such as from 10 to 15 conservative amino acid substitutions, for example from 15 to 25 conservative amino acid substtution. Conservative amino acid substitutions can be made within any one or more groups of predetermined amino acids as listed herein above under the section “Definitions”.

IGF-1, or a fragment thereof may comprise one or more conservative amino acid substitutions including one or more conservative amino acid substitutions within the same group of predetermined amino acids, or a plurality of conservative amino acid substitutions, wherein each conservative substitution is generated by substitution within a different group of predetermined amino acids.

Accordingly, variants of IGF-1, or a fragment thereof according to the invention may comprise at least one substitution, such as a plurality of substitutions introduced independently of one another. IGF-1, or a fragment thereof, may thus comprise conwervative substitutions independently of one another, wherein at least one glycine (Gly) of said IGF-1, or a fragment thereof, is substituted with an amino acid selected from the group of amino acids consisting of Ala, Val, Leu, and Ile, and independently thereof, substitution of at least one alanine (Ala) of said IGF-1, or a fragment thereof, with an amino acid selected from the group of amino acids consisting of Gly, Val, Leu, and Ile, and independently thereof, substitution of at least one valine (Val) of said IGF-1, or a fragment thereof, with an amino acid selected from the group of amino acids consisting of Gly, Ala, Leu, and Ile, and independently thereof, substitution of at least one leucine (Leu) of said IGF-1, or a fragment thereof, with an amino acid selected from the group of amino acids consisting of Gly, Ala, Val, and Ile, and independently thereof, substitution of at least one isoleucine (Ile) of said IGF-1, or a fragment thereof, with an amino acid selected from the group of amino acids consisting of Gly, Ala, Val and Leu, and independently thereof, substitution of at least one aspartic acid (Asp) of said IGF-1, or a fragment thereof, with an amino acid selected from the group of amino acids consisting of Glu, Asn, and Gin, and independently thereof, substitution of at least one phenylalanine (Phe) of said IGF-1, or a fragment thereof, with an amino acid selected from the group of amino acids consisting of Tyr, Trp, His, Pro, and preferably selected from the group of amino acids consisting of Tyr and Trp, and independently thereof, substitution of at least one tyrosine (Tyr) of said IGF-1, or a fragment thereof, with an amino acid selected from the group of amino acids consisting of Phe, Trp, His, Pro, preferably an amino acid selected from the group of amino acids consisting of Phe and Trp, and independently thereof, substitution of at least one arginine (Arg) of said IGF-1, or a fragment thereof, with an amino acid selected from the group of amino acids consisting of Lys and His, and independently thereof, substitution of at least one lysine (Lys) of said IGF-1, or a fragment thereof, with an amino acid selected from the group of amino acids consisting of Arg and His, and independently thereof, substitution of at least one asparagine (Asn) of said IGF-1, or a fragment thereof, with an amino acid selected from the group of amino acids consisting of Asp, Glu, and Gin, and independently thereof, substitution of at least one glutamine (Gln) of said IGF-1, or a fragment thereof, with an amino acid selected from the group of amino acids consisting of Asp, Glu, and Asn, and independently thereof, substitution of at least one proline (Pro) of said IGF-1, or a fragment thereof, with an amino acid selected from the group of amino acids consisting of Phe, Tyr, Trp, and His, and independently thereof, substitution of at least one cysteine (Cys) of said IGF-1, or a fragment thereof, with an amino acid selected from the group of amino acids consisting of Asp, Glu, Lys, Arg, His, Asn, Gin, Ser, Thr, and Tyr.

It is clear from the above outline that the same fragment may comprise more than one conservative amino acid substitution from more than one group of conservative amino acids as defined herein.

The addition or deletion of an amino acid may be an addition or deletion of from 2 to preferably 10 amino acids, such as from 2 to 8 amino acids, for example from 2 to 6 amino acids, such as from 2 to 4 amino acids. However, additions of more than 10 amino acids, such as additions from 10 to 200 amino acids, are also comprised within the present invention.

An IGF-1 variant, or a fragment thereof, according to the present invention, may in one embodiment comprise less than 75 amino acid residues, for example less than 70 amino acid residues, such as less than 65 amino acid residues, for example less than 60 amino acid residues, such as less than 55 amino acid residues, for example less than 50 amino acid residues, such as less than 45 amino acid residues, for example less than 40 amino acid residues, such as less than 35 amino acid residues, for example less than 30 amino acid residues, such as less than 25 amino acid residues, for example less than 20 amino acid residues, such as less than 15 amino acid residues, for example less than 10 amino acid residues.

Variants as used herein is preferably by means of reference to the corresponding functionality of a predetermined IGF-1 amino acid sequence, more preferably the sequence of the 70 amino acids of IGF.

One method of determining a sequence of immunogenically active amino acids within a known amino acid sequence has been described by Geysen in U.S. Pat No. 5,595,915 and is incorporated herein by reference.

A further suitably adaptable method for determining structure and function relationships of peptide fragments is described by U.S. Pat. No. 6,013,478, which is herein incorporated by reference. Also, methods of assaying the binding of an amino acid sequence to a receptor moiety are known to the skilled artisan.

Variants of IGF-1, or a fragment thereof will be understood to exhibit amino acid sequences gradually departing from the preferred, predetermined sequence of human IGF-1, as the number and scope of insertions, deletions and substitutions including conservative substitutions increases. This departure is measured as a reduction in homology between human IGF-1 and the variant.

All IGF-1 variants and fragments thereof are included within the scope of this invention, regardless of the degree of homology that they show to human IGF-1. The reason for this is that some regions of IGF-1 are most likely readily mutatable, or capable of being completely deleted, without any significant biological effect of the resulting fragment.

A functional variant obtained by substitution may well exhibit some form or degree of native IGF-1 activity, and yet be less homologous, if residues containing functionally similar amino acid side chains are substituted. Functionally similar in this respect refers to dominant characteristics of the side chains such as hydrophobic, basic, neutral or acidic, or the presence or absence of steric bulk. Accordingly, in one embodiment of the invention, the degree of identity between i) a given IGF-1 variant, or a fragment thereof, capable of binding an IGF-1 receptor moiety, and ii) a preferred predetermined fragment such as e.g. human IGF-1, is not a principal measure of the variant of a preferred predetermined IGF-1 sequence, such as e.g. human IGF-1.

IGF-1 variants, and fragments thereof, sharing at least some homology with a preferred predetermined IGF-1 molecule, preferably human IGF-1, or a fragment, are to be considered as falling within the scope of the present invention when such variants are at least about 40 percent homologous with human IGF-1, such as at least about 50 percent homologous, for example at least about 60 percent homologous, such as at least about 70 percent homologous, for example at least about 75 percent homologous, such as at least about 80 percent homologous, for example at least about 85 percent homologous, such as at least about 90 percent homologous, for example at least 92 percent homologous, such as at least 94 percent homologous, for example at least 95 percent homologous, such as at least 96 percent homologous, for example at least 97 percent homologous, such as at least 98 percent homologous, for example at least 99 percent homologous homologous with human IGF-1.

Homology may preferably be calculated by any suitable algorithm or by computerised implementations of such algorithms for example CLUSTAL in the PC/Gene program by Intelligenetics or GAP, BESTFIT, BLAST, FASTA and TFASTA in the Wisconsin Genetics Software Package, Genetics Computer Group (GCG). The homology between amino acid sequences may furthermore be calculated with the aid of well known matrices such as for example any one of BLOSUM 30, BLOSUM 40, BLOSUM 45, BLOSUM 50, BLOSUM 55, BLOSUM 60, BLOSUM 62, BLOSUM 65, BLOSUM 70, BLOSUM 75, BLOSUM 80, BLOSUM 85, and BLOSUM 90.

Additional factors that may be taken into consideration when determining variants according to the definition used herein are i) the ability of antisera which are capable of substantially neutralizing the binding of IGF-1 to an IGF-1 receptor moiety to detect an IGF-1 variant according to the present invention, and ii) the ability of the IGF-1 variant to compete with IGF-1 for a receptor moiety.

A non-conservative substitution leading to the formation of a variant IGF-1, or a fragment thereof, would for example i) differ substantially in hydrophobicity, for example a hydrophobic residue (Val, Ile, Leu, Phe or Met) substituted for a hydrophilic residue such as Arg, Lys, Trp or Asn, or a hydrophilic residue such as Thr, Ser, His, Gin, Asn, Lys, Asp, Glu or Trp substituted for a hydrophobic residue; and/or ii) differ substantially in its effect on polypeptide backbone orientation such as substitution of or for Pro or Gly by another residue; and/or iii) differ substantially in electric charge, for example substitution of a negatively charged residue such as Glu or Asp for a positively charged residue such as Lys, His or Arg (and vice versa); and/or iv) differ substantially in steric bulk, for example substitution of a bulky residue such as His, Trp, Phe or Tyr for one having a minor side chain, e.g. Ala, Gly or Ser (and vice versa).

In a further embodiment the present invention relates to IGF-1 variants, or a fragment thereof, wherein such variants comprise substituted amino acids having hydrophilic or hydropathic indices that are within +/−2.5, for example within +/−2.3, such as within +/−2.1, for example within +/−2.0, such as within +/−1.8, for example within +/−1.6, such as within +/−1.5, for example within +/−1.4, such as within +/−1.3 for example within +/−1.2, such as within +/−1.1, for example within +/−1.0, such as within +/−0.9, for example within +/−0.8, such as within +/−0.7, for example within +/−0.6, such as within +/−0.5, for example within +/−0.4, such as within +/−0.3, for example within +/−0.25, such as within +/−0.2 of the value of the amino acid it has substituted.

The importance of the hydrophilic and hydropathic amino acid indices in conferring interactive biologic function on a protein is well understood in the art (Kyte & Doolittie, 1982 and Hopp, U.S. Pat. No. 4,554,101, each incorporated herein by reference).

The amino acid hydropathic index values as used herein are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5) (Kyte & Doolittle, 1982).

The amino acid hydrophilicity values are: arginine (+3.0); lysine (+3.0); aspartate (+3.0.+−0.1); glutamate (+3.0.+−0.1); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4) (U.S. Pat. No. 4,554,101).

Substitution of amino acids can therefore in one embodiment be made based upon their hydrophobicity and hydrophilicity values and the relative similarity of the amino acid side-chain substituents, including charge, size, and the like. Exemplary amino acid substitutions which take various of the foregoing characteristics into consideration are well known to those of skill in the art and include: i) arginine and lysine; ii) glutamate and aspartate; iii) serine and threonine; iv) glutamine and asparagine; and v) valine, leucine and isoleucine.

In addition to the peptidyl compounds described herein, sterically similar compounds may be formulated to mimic the key portions of the peptide structure and such compounds may also be used in the same manner as the peptides of the invention. This may be achieved by techniques of modelling and chemical designing known to those of skill in the art. For example, esterification and other alkylations may be employed to modify the amino terminus of, e.g., a di-arginine peptide backbone, to mimic a tetra peptide structure. It will be understood that all such sterically similar constructs fall within the scope of the present invention.

Peptides with N-terminal alkylations and C-terminal esterifications are also encompassed within the present invention. Variants also comprise glycosylated and covalent or aggregative conjugates formed with the same or other IGF-1 fragments and/or IGF-1 molecules, including dimers or unrelated chemical moieties. Such variants are prepared by linkage of functionalities to groups which are found in fragment including at any one or both of the N- and C-termini, by means known in the art.

Variants may thus comprise fragments conjugated to aliphatic or acyl esters or amides of the carboxyl terminus, alkylamines or residues containing carboxyl side chains, e.g., conjugates to alkylamines at aspartic acid residues; O-acyl derivatives of hydroxyl group-containing residues and N-acyl derivatives of the amino terminal amino acid or amino-group containing residues, e.g. conjugates with fMet-Leu-Phe or immunogenic proteins. Derivatives of the acyl groups are selected from the group of alkyl-moieties (including C3 to C10 normal alkyl), thereby forming alkanoyl species, and carbocyclic or heterocyclic compounds, thereby forming aroyl species. The reactive groups preferably are difunctional compounds known per se for use in cross-linking proteins to insoluble matrices through reactive side groups.

Covalent or aggregative variants and derivatives thereof are useful as reagents in immunoassays or for affinity purification procedures. For example, a variant of IGF-1 according to the present invention may be insolubilized by covalent bonding to cyanogen bromide-activated Sepharose by methods known per se or adsorbed to polyolefin surfaces, either with or without glutaraldehyde cross-linking, for use in an assay or purification of anti-IGF-1 antibodies or novel cell surface receptors. Fragments may also be labelled with a detectable group, e.g., radioiodinated by the chloramine T procedure, covalently bound to rare earth chelates or conjugated to another fluorescent moiety for use in e.g. diagnostic assays.

Mutagenesis of IGF-1, or a fragment thereof, can be conducted by making amino acid insertions, usually on the order of about from 1 to 10 amino acid residues, preferably from about 1 to 5 amino acid residues, or deletions of from about from 1 to 10 residues, such as from about 2 to 5 residues.

In one embodiment the IGF-1, or a fragment thereof, is synthesised by automated synthesis. Any of the commercially available solid-phase techniques may be employed, such as the Merrifield solid phase synthesis method, in which amino acids are sequentially added to a growing amino acid chain. (See Merrifield, J. Am. Chem. Soc. 85:2149-2146, 1963). Equipment for automated synthesis of polypeptides is commercially available from suppllers such as Applled Biosystems, Inc. of Foster City, Calif., and may generally be operated according to the manufacturer's instructions. Solid phase synthesis will enable the incorporation of desirable amino acid substitutions into any IGF-1 variant, or a fragment thereof. It will be understood that substitutions, deletions, insertions or any subcombination thereof may be combined to arrive at a final sequence of a variant. Insertions shall be understood to include amino-terminal and/or carboxyl-terminal fusions, e.g. with a hydrophobic or immunogenic protein or a carrier such as any polypeptide or scaffold structure capable as serving as a carrier.

Oligomers including dimers including homodimers and heterodimers of IGF-1, or a fragment thereof, are also provided and fall under the scope of the invention. IGF-1-variants and variants can be produced as homodimers or heterodimers with other amino acid sequences or with native IGF-1 sequences.

Immunostimulating IGF-1 fragments according to the invention may be synthesised both in vitro and in vivo. Method for in vitro synthesis are well known, and methods being suitable or suitably adaptable to the synthesis in vivo of IGF-1 are also described in the prior art. When synthesized in vivo, a host cell is transformed with vectors containing DNA encoding the IGF-1 fragment it is desired to express. A vector is defined as a replicable nucleic acid construct. Vectors are used to mediate expression of the IGF-1 fragment. An expression vector is a replicable DNA construct in which a nucleic acid sequence encoding a predetermined IGF-1 sequence, or any variant thereof that can be expressed in vivo, is operably linked to suitable control sequences capable of effecting the expression in a suitable host. Such control sequences are well known in the art.

By analogy to what is described herein above it is also possible to provide IGF-1 binding protein variants that can be administered to an individual in combination with IGF1. IGF-1 binding protein variants are variants of IGF-1 binding proteins, or fragments thereof, comprising an amino acid sequence capable of being recognised by an antibody also capable of recognising an IGF-1 binding protein having a predetermined amino acid sequence, such as a native sequence, and/or variants of IGF-1 binding proteins, or fragments thereof, comprising an amino acid sequence capable of binding to IGF-1, preferably human IGF-1, and forming a complex therewith, and/or a variant of an IGF-1 binding protein exerting substantially similar agonistic or antagonistic effects.

### DETAILED DESCRIPTION OF THE INVENTION

In one embodiment there is provided a method of treatment of an individual, preferably a human being, suffering from a liver disease, or at risk of contracting a liver disease unless treated, said method comprising the step of administering to the individual a composition comprising a pharmaceutically effective amount of IGF-1, or a variant thereof.

The liver disease may be an acute liver disease or it may be a chronic liver disease. Examples of acute liver disease according to the invention are liver failure. The acute liver disease may occur in combination with malnutrition, in combination with with insulin resistance, or in combination with IGF-1 deficiency.

When the liver disease is a chronic liver disease it may e.g. be cirrhosis of the liver, fibrosis of the liver, or chronic hepatitis. The chronic liver disease may occur in combination with any one or more secondary conditions and indications such as e.g. a metabolic disorder, malnutrition, insulin resistance, diabetes mellitus, IGF-1 deficiency, hepatic encephalopathy, hepatic encephalopathy and ascites, portal hypertension, portal hypertension and ascites, hepatic nephropathy, as well as hepatic nephropathy and ascites.

IGF-1 deficiency as used herein signifies an individual having a serum concentration of circulating IGF-1 of less than about 180 microgram per litre, such as less than 175 microgram per litre, for example less than 170 microgram per litre, such as less than 165 microgram per litre, for example less than 160 microgram per litre, such as less than 155 microgram per litre, for example less than 150 microgram per litre, such as less than 145 microgram per litre, such as less than 140 microgram per litre, for example less than 135 microgram per litre, such as less than 130 microgram per litre, for example less than 125 microgram per litre, for example less than 120 microgram per litre, such as less than 115 microgram per litre, for example less than 110 microgram per litre, such as less than 105 microgram per litre, for example less than 100 microgram per litre such as less than 95 microgram per litre, for example less than 90 microgram per litre, such as less than 85 microgram per litre, for example less than 80 microgram per litre such as less than 75 microgram per litre, for example less than 70 microgram per litre, such as less than 65 microgram per litre, for example less than 60 microgram per litre, such as less than 55 microgram per litre, for example less than 50 microgram per litre, such as less than 45 microgram per litre, for example a serum concentration of circulating IGF-1 of less than 40 microgram per litre.

In one preferred aspect of the present invention there is provided a method of treatment of an individual, preferably a human being, that is deficient in IGF-1, or at risk of becoming deficient in IGF-1 unless treated, said method comprising the step of administering to the individual a composition comprising a pharmaceutically effective amount of IGF-1, or a variant thereof.

The method according to the invention is preferably selected from a method of treatment that is prophylactic, a method of treatment that is ameliorating, and a method of treatment that is curative.

The IGF-1 according to the invention, preferably comprised in a pharmaceutical composition, is preferably administrered to the individual to be treated by means of subcoutaneous injection, preferably once or twice a day. However other means of injection, such as e.g. running infusion or slow infusion may also be used, and injections more than twice a day can also be performed when there is a need for this.

The pharmaceutically effective amount of IGF-1 is preferably less than 1200 microgram per day per kilogram of treated individual, such as less than 1150 microgram per day per kilogram of treated individual, for example less than 1100 microgram per day per kilogram of treated individual, such as less than 1050 microgram per day per kilogram of treated individual, for example less than 1000 microgram per day per kilogram of treated individual, such as less than 950 microgram per day per kilogram of treated individual, for example less than 900 microgram per day per kilogram of treated individual, such as less than 850 microgram per day per kilogram of treated individual, for example less than 800 microgram per day per kilogram of treated individual, such as less than 750 microgram per day per kilogram of treated individual, for example less than 700 microgram per day per kilogram of treated individual, such as less than 650 microgram per day per kilogram of treated individual, for example less than 600 microgram per day per kilogram of treated individual, such as less than 550 microgram per day per kilogram of treated individual, for example less than 500 microgram per day per kilogram of treated individual, such as less than 480 microgram per day per kilogram of treated individual, for example less than 460 microgram per day per kilogram of treated individual, such as less than 440 microgram per day per kilogram of treated individual, for example less than 420 microgram per day per kilogram of treated individual, such as less than 400 microgram per day per kilogram of treated individual, for example less than 380 microgram per day per kilogram of treated individual, such as less than 360 microgram per day per kilogram of treated individual, for example less than 340 microgram per day per kilogram of treated individual, such as less than 320 microgram per day per kilogram of treated individual, for example less than 300 microgram per day per kilogram of treated individual, such as less than 290 microgram per day per kilogram of treated individual, for example less than 280 microgram per day per kilogram of treated individual, such as less than 270 microgram per day per kilogram of treated individual, for example less than 260 microgram per day per kilogram of treated individual, such as less than 250 microgram per day per kilogram of treated individual, for example less than 1000 microgram per day per kilogram of treated individual, such as less than 240 microgram per day per kilogram of treated individual, for example less than 230 microgram per day per kilogram of treated individual, such as less than 220 microgram per day per kilogram of treated individual, for example less than 210 microgram per day per kilogram of treated individual, such as less than 200 microgram per day per kilogram of treated individual, for example less than 190 microgram per day per kilogram of treated individual, such as less than 180 microgram per day per kilogram of treated individual, for example less than 170 microgram per day per kilogram of treated individual, such as less than 160 microgram per day per kilogram of treated individual, for example less than 150 microgram per day per kilogram of treated individual, such as less than 145 microgram per day per kilogram of treated individual, for example less than 140 microgram per day per kilogram of treated individual, such as less than 135 microgram per day per kilogram of treated individual, for example less than 130 microgram per day per kilogram of treated individual, such as less than 125 microgram per day per kilogram of treated individual, for example less than 120 microgram per day per kilogram of treated individual, such as less than 115 microgram per day per kilogram of treated individual, for example less than 110 microgram per day per kilogram of treated individual, such as less than 105 microgram per day per kilogram of treated individual, for example about 100 microgram per day per kilogram of treated individual, for example less than about 100 microgram per day per kilogram of treated individual, such as less than 95 microgram per day per kilogram of treated individual, for example less than 90 microgram per day per kilogram of treated individual, such as less than 85 microgram per day per kilogram of treated individual, for example less than 80 microgram per day per kilogram of treated individual, such as less than 75 microgram per day per kilogram of treated individual, for example less than 70 microgram per day per kilogram of treated individual, such as less than 65 microgram per day per kilogram of treated individual for example less than 60 microgram per day per kilogram of treated individual, such as less than 55 microgram per day per kilogram of treated individual, for example less than about 50 microgram per day per kilogram of treated individual, and preferably more than about 25 microgram per day per kilogram of treated individual, such as more than about 30 microgram per day per kilogram of treated individual, for example more than about 35 microgram per day per kilogram of treated individual, such as more than about 40 microgram per day per kilogram of treated individual.

In one embodiment the pharmaceutically effective amount of IGF-1 is administrered to the individual to be treated once or twice a day by subcutaneous injection at least during prolonged periods of an essentially life-long treatment regime. Prolonged periods may range from several weeks to several months to several years.

It is desirable to reduce the period of treatment, and in one embodiment the pharmaceutically effective amount of IGF-1 is administrered to the individual to be treated once or twice a day by subcutaneous injection for a period of treatment lasting less than 6 months, such as less than 5 months, for example less than 4 months, such as less than 3 months, for example less than 2 months, such as less than 1 month, for example less than 2 weeks, such as a treatment period of about 1 week.

In one preferred embodiment the composition is administrered to the individual prior to, during, or after liver transplantation treatment.

IGF-1 according to the invention can be isolated from any source including any source capable of producing said IGF-1 by recombinant DNA techniques. Recombinant IGF-1, or a variant thereof, produced by recombinant DNA technology is particularly preferred according to the present invention. The recombinant IGF-1 is preferably recombinant human IGF-1, or a variant thereof.

The pharmaceutical composition according to the invention may further comprise at least one IGF-1 binding protein (IGFBP) selected from the group consisting of IGFBP-1, IGFBP-2, IGFBP-3, IGFBP-4, IGFBP-5, and IGFBP-6, including any variant thereof. A composition further comprising IGFBP-3, or a variant thereof is preferred.

It is much preferred that the composition comprises a complex formed of at least some of said IGF-1 and some of said IGFBP-3 comprised in the composition. The composition in one embodiment may further comprise acid labile subunit (ALS) capable of forming a complex with IGF-1 and IGFBP-3. In another preferred embodiment the composition further comprises a protease inhibitor capable of inhibiting proteases having an affinity for IGF-1.

When a variant of IGF-1 is used, such a variant is in one embodiment a variant comprising at least one conservative amino acid substitution, such as a plurality of conservative amino acid substitutions. The variant may be at least 96 percent identical or homologous homologous to human IGF-1, such as at least 98 percent identical or homologous to IGF-1.

The composition may further comprise a pharmaceutically acceptable carrier such as e.g. a sterile, isotonic solution containing a citrate buffer of pH about 6.

In a number of preferred embodiments the present invention provides treatment of specific conditions present in the individual treated according to the invention.

Such specific treatments of an individual suffering from an acute or chronic liver disease are described herein below and involve, but is not limited to, methods comprising:

Treating insulin resistence in an individual suffering from acute or chronic liver disease, said method comprising the step of administering to the individual a composition comprising a pharmaceutically effective amount of IGF-1, or a variant thereof.

Treating diabetes mellitus in in an individual suffering from acute or chronic liver disease, said method comprising the step of administering to the individual a composition comprising a pharmaceutically effective amount of IGF-1, or a variant thereof.

Treating diabetes mellitus and insulin resistence in an individual suffering from acute or chronic liver disease, said method comprising the step of administering to the individual a composition comprising a pharmaceutically effective amount of IGF-1, or a variant thereof.

Treating hyperinsulinaemiae in in an individual suffering from acute or chronic liver disease, said method comprising the step of administering to the individual a composition comprising a pharmaceutically effective amount of IGF-1, or a variant thereof.

Treating hyperaminoacidemiae in an individual suffering from a chronic liver disease, said method comprising the step of administering to the individual a composition comprising a pharmaceutically effective amount of IGF-1, or a variant thereof.

Treating hyperinsulinaemiae and hyperaminoacidemiae in an individual suffering from acute or chronic liver disease, said method comprising the step of administering to the individual a composition comprising a pharmaceutically effective amount of IGF-1, or a variant thereof.

Treating hyperaminoacidemiae and reducing muscle proteolysis in an individual suffering from a chronic liver disease, said method comprising the step of administering to the individual a composition comprising a pharmaceutically effective amount of IGF-1, or a variant thereof.

Treating a metabolic disorder in an individual suffering from acute or chronic liver disease, said method comprising the step of administering to the individual a composition comprising a pharmaceutically effective amount of IGF-1, or a variant thereof.

Treating malnutrition in an individual suffering from acute or chronic liver disease, said method comprising the step of administering to the individual a composition comprising a pharmaceutically effective amount of IGF-1, or a variant thereof.

Treating malnutrition and a metabolic disorder in an individual suffering from acute or chronic liver disease, said method comprising the step of administering to the individual a composition comprising a pharmaceutically effective amount of IGF-1, or a variant thereof.

Restoring normal physiological serum levels of IGF-1 in an individual suffering from acute or chronic liver disease, said method comprising the step of treating the individual by administering to the individual a composition comprising a pharmaceutically effective amount of IGF-1, or a variant thereof.

Treating hepatic encephalopathy in an individual suffering from acute or chronic liver disease, said method comprising the step of administering to the individual a composition comprising a pharmaceutically effective amount of IGF-1, or a variant thereof.

Treating hepatic encephalopathy in combination with ascites in an individual suffering from acute or chronic liver disease, said method comprising the step of administering to the individual a composition comprising a pharmaceutically effective amount of IGF-1, or a variant thereof.

Treating hepatic nephropathy in an individual suffering from acute or chronic liver disease, said method comprising the step of administering to the individual a composition comprising a pharmaceutically effective amount of IGF-1, or a variant thereof.

Treating hepatic nephropathy in combination with ascites in an individual suffering from acute or chronic liver disease, said method comprising the step of administering to the individual a composition comprising a pharmaceutically effective amount of IGF-1, or a variant thereof.

Treating portal hypertension in an individual suffering from acute or chronic liver disease, said method comprising the step of administering to the individual a composition comprising a pharmaceutically effective amount of IGF-1, or a variant thereof.

Treating portal hypertension in combination with ascites in an individual suffering from acute or chronic liver disease, said method comprising the step of administering to the individual a composition comprising a pharmaceutically effective amount of IGF-1, or a variant thereof.

Treating hepatic encephalopathy and hepatic nephropathy in an individual suffering from acute or chronic liver disease, said method comprising the step of administering to the individual a composition comprising a pharmaceutically effective amount of IGF-1, or a variant thereof.

Treating hepatic encephalopathy and hepatic nephropathy in combination with ascites an individual suffering from acute or chronic liver disease, said method comprising the step of administering to the individual a composition comprising a pharmaceutically effective amount of IGF-1, or a variant thereof.

Treating hepatic encephalopathy and portal hypertension in an individual suffering from acute or chronic liver disease, said method comprising the step of administering to the individual a composition comprising a pharmaceutically effective amount of IGF-1, or a variant thereof.

Treating hepatic encephalopathy and portal hypertension in combination with ascites in an individual suffering from acute or chronic liver disease, said method comprising the step of administering to the individual a composition comprising a pharmaceutically effective amount of IGF-1, or a variant thereof.

Treating hepatic nephropathy and portal hypertension in an individual suffering from acute or chronic liver disease, said method comprising the step of administering to the individual a composition comprising a pharmaceutically effective amount of IGF-1, or a variant thereof.

Treating hepatic nephropathy and portal hypertension in an individual suffering from acute or chronic liver disease, said method comprising the step of administering to the individual a composition comprising a pharmaceutically effective amount of IGF-1, or a variant thereof.

Treating hepatic nephropathy and portal hypertension in an individual suffering from acute or chronic liver disease, said method comprising the step of administering to the individual a composition comprising a pharmaceutically effective amount of IGF-1, or a variant thereof.

Treating hepatic nephropathy and portal hypertension in combination with ascites in an individual suffering from acute or chronic liver disease, said method comprising the step of administering to the individual a composition comprising a pharmaceutically effective amount of IGF-1, or a variant thereof.

Treating hepatic encephalopathy and hepatic nephropathy and portal hypertension in an individual suffering from acute or chronic liver disease, said method comprising the step of administering to the individual a composition comprising a pharmaceutically effective amount of IGF-1, or a variant thereof.

Treating hepatic encephalopathy and hepatic nephropathy and portal hypertension in combination with ascites in an individual suffering from acute or chronic liver disease, said method comprising the step of administering to the individual a composition comprising a pharmaceutically effective amount of IGF-1, or a variant thereof.

Reducing serum glucose concentrations in an individual suffering from acute or chronic liver disease, said method comprising the step of treating the individual by administering to the individual a composition comprising a pharmaceutically effective amount of IGF-1, or a variant thereof.

Increasing hepatic amino acid conversion in an individual suffering from acute or chronic liver disease, said method comprising the step of treating the individual by administering to the individual a composition comprising a pharmaceutically effective amount of IGF-1, or a variant thereof.

Reducing serum glucose concentrations and increasing hepatic amino acid conversion in an individual suffering from acute or chronic liver disease, said method comprising the step of treating the individual by administering to the individual a composition comprising a pharmaceutically effective amount of IGF-1, or a variant thereof.

Reducing muscle proteolysis in an individual suffering from a chronic liver disease, said method comprising the step of treating the individual by administering to the individual a composition comprising a pharmaceutically effective amount of IGF-1, or a variant thereof.

Reducing increased levels of growth hormone in an individual suffering from a chronic liver disease, said method comprising the step of treating the individual by administering to the individual a composition comprising a pharmaceutically effective amount of IGF-1, or a variant thereof.

In another aspect of the invention there is provided IGF-1, or a composition comprising IGF-1, or a variant thereof, for use in any method of the invention.

The composition preferably comprises an IGF-1 produced by recombinant DNA technology, more preferably recombinant human IGF-1.

The composition may further comprise at least one IGF-1 binding protein (IGFBP) selected from the group consisting of IGFBP-1, IGFBP-2, IGFBP-3, IGFBP-4, IGFBP-5, and IGFBP-6, including any variant thereof. More preferred the composition further comprises IGFBP-3, or a variant thereof. A complex is preferably formed of at least some of said IGF-1 and some of said IGFBP-3 comprised in the composition that may even further comprise acid labile subunit (ALS) capable for forming a complex with IGF-1 and IGFBP-3.

To avoid IGF-1 proteolysis the composition may comprise a protease inhibitor capable of inhibiting proteases having an affinity for IGF-1.

When a variant of IGF-1 is comprised in the composition it is preferably a variant comprising at least one conservative amino acid substitution, or an IGF-1 variant that is at least 98 percent homologous to human IGF-1.

The composition may further comprise any pharmaceutically acceptable carrier.

In yet another aspect there is provided the use of IGF-1, or a variant thereof, for the manufacture of a medicament for treatment of acute or chronic liver disease in an individual, preferably a human being, in need of said treatment.

There is also provided the use of a composition according to the invention for the manufacture of a medicament for treatment of acute or chronic liver disease in an individual, preferably a human being, in need of said treatment.

The liver disease may be any acute liver disease or any chronic liver disease as described herein above, and the liver disease may occur in combination with any secondary condition or indication referred to herein above.

The treatment in question may be prophylactic, ameliorating or curative.

The treatment may involve administration of the medicament by means of subcutaneous injection, preferably once or twice a day. The pharmaceutically effective amount of IGF-1 is as stated herein above, and the period of treatment is also as stated herein above.

Administration of compositions comprising IGF-1

IGF-1 is directly administered to an individual including a human being by any suitable technique, including parenteral administration, and can be administered locally or systemically. The specific route of administration will depend, e.g., on the medical history of the patient, including any perceived or anticipated side effects using IGF-1. Examples of parenteral administration include subcutaneous, intramuscular, intravenous, intraarterial, and intraperitoneal administration.

Preferably, the administration is by continuous infusion (using, e.g., minipumps such as osmotic pumps and a subcutaneous route), or by a single injection or multiple (e.g., 2-4) injections using, e.g., intravenous or subcutaneous means. Preferably, the administration is subcutaneous for IGF-1. The administration may also be as a single bolus or by slow-release depot formulation.

In addition, the IGF-1 is suitably administered together with any one or more of its binding proteins, for example, those currently known, i.e., IGFBP-1, IGFBP 2, IGFBP-3, IGFBP-4, IGFBP-5, or IGFBP-6. The preferred binding protein for IGF-1 administration in accordance with the invention is IGFBP-3, which is described in WO 89/09268 published Oct. 5, 1989 and by Martin and Baxter, J. Biol. Chem., 261. 8754-8760 (1986). This glycosylated IGFBP-3 protein is an acid-stable component of about 53 Kd on a non-reducing SDS-PAGE gel of a 125-150 Kd glycoprotein complex found in human plasma that carries most endogenous IGF. The IGF-1 may also be suitably coupled to a receptor or antibody or antibody fragment for administration.

The administration of the IGF binding protein with IGF-1 is in one embodiment accomplished by the method described in U.S. Pat. No. 5,187,151, the disclosure of which is incorporated herein by reference. Briefly, the IGF-1 and IGFBP are administered in effective amounts by subcutaneous bolus injection in a molar ratio of from about 0.5:1 to about 3:1, preferably about 1:1.

The IGF-1 composition to be used in the therapy will be formulated and dosed in a fashion consistent with good medical practice, taking into account the clinical condition of the individual patient (especially the side effects of treatment with IGF-1 alone), the site of delivery of the IGF-1 composition, the method of administration, the scheduling of administration, and other factors known to practitioners. The “effective amount” of IGF-1 for purposes herein is thus determined by such considerations.

As a general proposition, the total pharmaceutically effective amount of the IGF-1 administered parenterally will be in the range of daily doses of from about 50 microgram per kilogram body weight of the individual to which the IGF-1 is administered (i.e. 50 μg IGF-1/kg/day) to less than about 1200 μg/kg/day of patient body weight, although, as noted above, this will be subject to therapeutic discretion. More preferably, this dose is at least 75 μg/kg/day, and most preferably for humans between about 100 and 300 μg/kg/day.

If given continuously or semi-continuously the IGF-1 is typically administered at a dose rate of about 1 μg/kg/hour to about 50 μg/kg/hour, either by 1-4 bolus injections or per day, by running infusion, or by slow infusion such as by continuous subcutaneous infusions using, for example, a mini-pump or a drop. An intravenous bag solution may also be employed. The key factor in selecting an appropriate dose is the result obtained, as measured at least by amelioration of the acute or chronic liver disease in question.

The IGF-1 composition according to the invention may also be administered by sustained-release systems. Suitable examples of sustained-release compositions include semi-permeable polymer matrices in the form of shaped articles, e.g., films, or microcapsules. Sustained-release matrices include polylactides (U.S. Pat. No. 3,773,919, EP 58,481), copolymers of L-glutamic acid and gamma-ethyl-L-glutamate (Sidman et al., Biocolymers, 22, 547-556 [1983]), poly(2-hydroxyethyl methacrylate) (Langer et al., J. Biomed. Mater. Res., 15: 167-277 [1981], and Langer, Chem. Tech., 12: 98-105 [1982]), ethylene vinyl acetate (Langer et al., supra) or poly-D-(−)-3-hydroxybutyric acid (EP 133,988). Sustained-release IGF-1 compositions also include liposomally entrapped IGF-1. Liposomes containing IGF-1 are prepared by methods known per se: DE 3,218,121; Epstein et al., Proc. Natl. Acad. Sci. U.S.A., 82: 3688-3692 (1985); Hwang et al., Proc. Natl. Acad. Sci. U.S.A., 77: 4030-4034 (1980); EP 52,322; EP 36,676; EP 88,046; EP 143,949; EP 142,641; Japanese Pat. Appln. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal IGF-1 therapy.

For parenteral administration, in one embodiment, the IGF-1 is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting the IGF-1 uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or nonionic surfactants such as polysorbates, polyoxamers, or PEG.

The IGF-1 is typically formulated in such vehicles at a concentration of about 0.1 mg/ml to 100 mg/ml, preferably about 1 to 10 mg/ml, at a pH of about 3 to 8. Full-length IGF-1 is generally stable at a pH of no more than about 6; des(1-3) IGF-1 is stable at about 3.2 to 5. It will be understood that use of certain of the foregoing excipients, carriers, or stabilizers will result in the formation of IGF-1 salts.

In addition, the IGF-1, preferably the full-length IGF-1, is suitably formulated in an acceptable carrier vehicle to form a pharmaceutical composition, preferably one that does not contain cells. Recombinant, full length IGF-1, preferably recombinant, full length human IGF-1, or a variant thereof, is preferred for such a composition. In one embodiment, the buffer used for formulation will depend on whether the composition will be employed immediately upon mixing or stored for later use. If employed immediately, the full-length IGF-1 can be formulated in mannitol, glycine, and phosphate, pH 7.4. If this mixture is to be stored, it is formulated in a buffer at a pH of about 6, such as citrate, with a surfactant that increases the solubility of the IGF-1 at this pH, such as 0.1% polysorbate 20 or poloxamer 188. The final preparation may be a stable liquid or lyophilized solid.

IGF-1 to be used for therapeutic administration must be sterile. Sterility is readily accomplished by filtration through sterile filtration membranes (e.g., 0.2 micron membranes). Therapeutic IGF-1 compositions generally are placed into a container having a sterile access port, for example, an intravenous solution bag or vial having a stopper pierceable by a hypodermic injection needle.

IGF-1 ordinarily will be stored in unit or multi-dose containers, for example, sealed ampoules or vials, as an aqueous solution, or as a lyophilized formulation for reconstitution. As an example of a lyophilized formulation, 10-ml vials are filled with 5 ml of sterile-filtered 1% (w/v) aqueous IGF-1 solution, and the resulting mixture is lyophilized. The infusion solution is prepared by reconstituting the lyophilized IGF-1 using bacteriostatic Water-for-Injection.

Effect of Treatment

In one embodiment of the present invention the treatment may result in a change of biochemical values indicative of hepatic disease. Preferably, the biochemical values may be improved.

Biochemical values may for example be selected from the group consisting of albumin, coagulation factor 3, 7 and 10 (INR), alanine aminotransferase (ALAT), aspartate aminotransferase (ASAT) and γ-glutamyl transferase (GGT) for example measured in blood samples.

In one embodiment the treatment results in an increase in substrate exchange, for example forearm substrate exchange. The substrate may for example be glucose or amino-N. The increase in substrate exchange may for example be determined as described in the examples.

The increase may for example be 1.1 fold, such as 1.1 to 1.2 fold, for example 1.2 to 1.5 fold, such as 1.5 to 2 fold, for example 2 to 3 fold, such as 3 to 5 fold. Preferably, however the increase is more than 1.5 fold.

### LITERATURE REFERENCES

¹Mann F C, Magath T B: Studies on the physiology of the liver. II. The effect of the removal of the liver on the blood sugar level. Arch Intern Med 30:73-84, 1922.

²Mann F C, Magath T B: Studies on the physiology of the liver. IV. The effect of total removal of the liver after pancreatectomy on the blood sugar level. Arch Intern Med 31:797-806, 1923.

³Bjornstorp P, Sjostrom L: Carbohydrate storage in man: speculations and some quantitative considerations. Metabolism 27 (Suppl. 2):1853-65, 1978.

⁴Katz L D, Glickman M G, Rapaport S, Ferrannini E, De Fronzo R A: Splanchnic and peripheral disposal of oral glucose in man. Diabetes 32:675-79, 1983.

⁵Karem J H, Forsham P H: Pancreatic hormones and diabetes mellitus. In Basic and Clinical Endocrinology. 4th edition. Greenspan F S, Baxter J D, Eds. Norwalk, Conn., Appleton and Lange, 1994, p. 571-634.

⁶McGilvery R W, Goldstein G: Biochemistry: a Functional Approach. 2nd edition. Philadelphia, Pa., W. B. Saunders, 1979.

⁷Scofield R F, Kosugi K, Schumann W C, Kumaran K, Landau B R: Quantitative estimation of the pathways followed in the conversion to glycogen of glucose administered to the fasted rat. J Biol Chem 260:8777-82, 1985.

⁸Consoli A, Nurjhan N, Capani F, Gerich J: Predominant role of gluconeogenesis in increased hepatic glucose production in NIDDM. Diabetes 38:550-57, 1989.

⁹Stone B E, VanThiel D H: Diabetes mellitus and the liver. Sem Liver Dis 5:8-28, 1985.

¹⁰Ferrannini E, Lanfranchi A, Rohner-Jeanrenaud F, Manfredini G, VandeWerve G: Influence of long-term diabetes on liver glycogen metabolism in the rat. Metabolism 39:1082-88, 1990.

¹¹Bogoch A, Casselman W G B, Kaplan A, Bockus H L: Studies of hepatic function in diabetes mellitus, portal cirrhosis and other liver diseases. Am J Med 18:354-84, 1955.

¹²Hildes J A, Sherlock S, Walshe V: Liver and muscle glycogen in normal subjects, in diabetes mellitus and in acute hepatitis. Clin Sci 7:289-95, 1949.

¹³Manderson W G, McKiddle M T, Manners D J, Stark J R: Liver glycogen accumulation in unstable diabetes. Diabetes 17:13-16, 1968.

¹⁴Ehrlich P: <ber das Vorkommen von Glykogen im diabetischen und normalen Organismus. Z Klin Med 6:33-53,1883.

¹⁵Kautzsch E: Leberbefunde bei diabetes mellitus. Med Mschr 17:229-34, 1963.

¹⁶Robbers H, Stohfeldt P, Kr;ger C: Differential diagnose de diabetischen und alkoholischen fettleber: untersuchungen and 171 Diabetikern und 100 patienten mit alkoholabusus. Deutsch Med Wschr 93:112-13, 1968.

¹⁷Creutzfeldt W, Frerichs H, Sickinger K: Liver diseases and diabetes mellitus. Prog Liver Dis 13:371-407, 1970.

¹⁸Kalk H: <ber die beziehungen zwischen Fettleber und diabetes. Deutsch Med Wschr 84:1898-1901, 1959.

¹⁹Kalk H: Über die Fettleber. Munchen Med Wschr 107:1141-47, 1965.

²⁰Chatila R, West A B: Hepatomegaly and abnormal liver tests due to glycogenesis in adults with diabetes. Med Balt 75:327-33, 1996.

²¹Leevy C M, Ryan C M, Fineberg J C: Diabetes mellitus and liver dysfunction. Am J Med 8:290-99, 1950.

²²Silverman J F, O'Brien K F, Long S, Leggett N, Khazanie P G, Pories W J, Norris J R, Caro J F: Liver pathology in morbidly obese patients with and without diabetes. Am J Gastroenterol 85:1349-55, 1990.

²³Jaques W E: The incidence of portal cirrhosis and fatty metamorphosis in patients dying with diabetes mellitus. N Engl J Med 249:442-45, 1953.

²⁴Bernuau D, Guillot R, Durand-Schneider A, Poussier P, Moreau P, Feldmann G: Liver perisinsoidal fibrosis in BB rats with or without overt diabetes. Am J Pathol 120:38-45, 1985.

²⁵Zimmerman H J, MacMurray F G, Rappaport H, Alpert L K: Studies of the liver in diabetes mellitus, II. J Lab Clin Med 36:922-27, 1950.

²⁶Leevy C M: Fatty liver: a study of 270 patients with biopsy-proven fatty liver and a review of the literature. Med Balt 41:249-76, 1962.

²⁷Connor B J F, Katbamna B, Tavill A S: Nonalcoholic fatty liver (NASH syndrome). Gastroenterologist 5:316-29, 1997.

²⁸Schaffner F, Thaler H: Nonalcoholic fatty liver disease. Prog Liver Dis 8:283-98, 1986.

²⁹Scatarige J C, Scott W W, Donovan P J, Siegelman S S, Sanders R C: Fatty infiltration of the liver: ultrasonographic and computerized tomographic correlation. J Ultrasound Med 3:9-14,1984.

³⁰Bacon B R, Farahvash M J, Janney C G, Neuschwander-Tetri B A: Nonalcoholic steatohepatitis: an expanded clinical entity. Gastroenterology 107:1103-09, 1994.

³¹Wanless J R, Lentz J S: Fatty liver hepatitis (steatohepatitis) and obesity: an autopsy study with analysis of risk factors. Hepatology 12:1106-10, 1990.

³²Kim W R, Poterucha J J, Porayko M K, Dickson E R, Steers J L, Wiesner R H: Recurrence of nonalcoholic steatohepatitis following liver transplantation. Transplantation 62:1802-1805, 1996.

³³Sheth S G, Gordon F D, Chopra S: Nonalcoholic steatohepatitis. Ann Intern Med 126:137-45, 1997.

³⁴Anderson T, Gluud C: Liver morphology in morbid obesity: a literature study. Int J Obesity 8:97-106, 1984.

³⁵Kern W H, Heger A H, Payne J H, DeWind L T: Fatty metamorphosis of the liver in morbid obesity. Arch Pathol 96:342-346,1973.

³⁶Nasrallah S M, Wills C E, Glambos J T: Hepatic morphology in obesity. Digest Dis Sci 26:325-27, 1981.

³⁷Falchuk K, Fiske S C, Haggit R C, Federman M, Trey C: Pericentral hepatic fibrosis and intracellular hyalin in diabetes mellitus. Gastroenterology 78:535-41, 1980.

³⁸Nagore N, Scheuer P: The pathology of diabetic hepatitis. J Pathol 156:155-60, 1988.

³⁹Bernuau D, Guillot R, Durand A, Raoux N, Gabreau T, Passa P, Feldmann G: Ultrastructural aspects of the liver perisinusoidal space in diabetic patients with and without microangiopathy. Diabetes 31:1061-67, 1982.

⁴⁰Ludwig J, McGill D B, Lindor K D: Review: nonalcoholic steatohepatitis. J Gastroenterol Hepatol 12:398-403, 1997.

⁴¹Braillon A, Capron J P, Herve M A, Degott C, Quenum C: Liver in obesity. Gut 26:133-39, 1985.

⁴²Palmer M, Schaffner F: The effect of weight reduction on hepatic abnormalities in overweight patients. Gastroenterology 99:1408-13, 1990.

⁴³Schlomerich J, Becher M S, Schmidt K, Schubert R, Kremer B, Feldhaus S, Gerok W: Influence of hydroxylation and conjugation of bile salts on their membranedamaging properties: studies on isolated hepatocytes and lipid membrane vesicles. Hepatology 4:661-66, 1984.

⁴⁴Abdelmalek M, Ludwig J, Lindor K D: Two cases from the spectrum of nonalcoholic steatohepatitis. J Clin Gastroenterol 20:127-30, 1995.

⁴⁵Laurin J, Lindor K D, Crippin J S, Gossard A, Gores G J, Ludwig J, Rakala J, McGill D B: Ursodeoxycholic acid or clofibrate in the treatment of non-alcoholic-induced steatohepatitis: a pilot study. Hepatology 23:1464-67, 1996.

⁴⁶Zimmerman H J, MacMurray F G, Rappaport H, Alpert L K: Studies of the liver in diabetes mellitus. J Lab Clin Med 36:912-21, 1950.

⁴⁷Hano T: Pathohistological study on the liver cirrhosis in diabetes mellitus. Kobe J Med Sci 14:87-106, 1968.

⁴⁸Warren S, LeCompte P M: The gall bladder. In The Pathology to Diabetes Mellitus. Warren S, LeCompte P M, Eds. Philadelphia, Lea & Febiger, 1952, 107-111.

⁴⁹Foster K J, Griffith A H, Dewbury K, Price C P, Wright R: Liver disease in patients with diabetes mellitus. Postgrad Med J 56:767-72, 1980.

⁵⁰Yang O, Arem R, Chan L: Gastrointestinal tract complications of diabetes mellitus. Arch Intern Med 144:1251-56,1984.

⁵¹Honore L H: The lack of a positive association between symptomatic cholesterol cholelithiasis and clinical diabetes mellitus: a retrospective study. J Chronic Dis 33:465-69, 1980.

⁵²Feldman M, Feldman M Jr: The incidence of cholelithiasis, cholesterosis, and liver disease in diabetes mellitus. Diabetes 3:305-307,1954.

⁵³Bennion L J, Grundy S M: Risk factors for the development of cholelithiasis. N Engl J Med 299:1161-67, 1978.

⁵⁴Cooper A D: Metabolic basis of cholesterol gallstone disease. Gastroenterol Clin North Am 20:21-46, 1991.

⁵⁵Ransohoff D F, Miller G L, Forsythe S B, Hermann R E: Outcome of acute cholecystitis in patients with diabetes mellitus. Ann Intern Med 106:829-32, 1987.

⁵⁶Onate-Ocana L F, Mondragon-Sanchez R J, Ruiz-Molina J M, Aiello-Crocifoglio V: Laparoscopic cholecystectomy at an oncologic center: a comparative analysis (Abstract). Rev Gastroenterol Med 32:101-107, 1997.

⁵⁷Physicians Desk Reference, 51st edition. Montvale, N.J., Medical Economics, 1997.

⁵⁸Bloodworth J M B, Hamwi G J: Histopathologic lesion associated with sulfonylurea administration. Diabetes 10:90-99, 1961.

⁵⁹Saw D, Pitman E, Maung M, Savasatit P, Wasserman D, Yeung C K: Granulomatous hepatitis associated with glyburide. Digest Dis Sci 41:322-25, 1996.

⁶⁰Sulkin T V, Bosman D, Krentz A J: Contraindications to metformin therapy in patients with NIDDM. Diabetes Care 20:925-28, 1997.

⁶¹Nosocomial hepatitis B virus infection associated with reusable fingerstick blood sampling devices—Ohio and New York City, 1996. Morb Mortal Wkly Rep 46:217-21, 1997.

62Polish L B, Shapiro C N, Bauer F, Klotz P, Ginier P, Roberto P R, Margolis H S, Alter M J: Nosocomial transmission of hepatitis B virus associated with the use of a spring-loaded finger-stick device. N Engl J Med 356:721-25, 1992.

⁶³Grimbert S, Valensi P, Levy-Marchal C, Perret G, Richardet J P, Raffoux C, Trinchet J C, Beaugrand M: High prevalence of diabetes mellitus in patients with chronic hepatitic C: a case-control study. Gastroenterol Clin Biol 20:544-48, 1996.

⁶⁴Fraser G M, Harman I, Meller N, Niv Y, Porath A: Diabetes mellitus is associated with chronic hepatitis C but not chronic hepatitis B infection. Isr J Med Sci 32:526-30, 1996.

⁶⁵Allison M E, Wreghitt T, Palmer C R, Alexander G J: Evidence for a link between hepatitis C virus infection and diabetes mellitus in a cirrhotic population. J Hepatol 21:1135-39, 1994.

⁶⁶Ozyilkan E, Arslan M: Increased prevalence of diabetes mellitus in patients with chronic hepatitis C virus infection. Am J Gastroenterol 91:1480-81, 1996.

⁶⁷Gray H, Wregitt T, Stratton I M, Alexander G J, Turner R C, O'Rahilly S: High prevalence of hepatitis C infection in Afro-Caribbean patients with type 2 diabetes and abnormal liver function tests. Diabetic Med 12:244-49, 1995.

⁶⁸Simo R, Hernadez C, Genesca J, Jardi R, Mesa J: High prevalence of hepatitis C virus infection in diabetic patients. Diabetes Care 19:998-1000, 1996.

⁶⁹Knobler H, Stagnaro-Green A, Wallenstein S, Schwartz M, Roman S H: Higher incidence of diabetes in liver transplant recipients with hepatitis C. J Clin Gastroenterol 26:30-33, 1998.

⁷⁰Chedin P, Cahen-Varsauz J, Boyer N: Non-insulin-dependent diabetes meilitus developing during interferon-alpha therapy for chronic hepatitis C. Ann Intern Med 125:521, 1996.

⁷¹Campbell S, McLaren E H, Danesh B J: Rapidly reversible increase in insulin requirement with interferon. Br Med J 313:92, 1996.

⁷²Shiba T, Morino Y, Tagawa K, Fujino H, Unuma T: Onset of diabetes with high titer anti-GAD antibody after IFN therapy for chronic hepatitis. Diabetes Res Clin Pract 30:237-41, 1995.

⁷³Lopes E P, Oliveira P M, Silva A E, Ferraz M L, Costa C H, Miranda W, Dib S A: Exacerbation of type 2 diabetes mellitus during interferon-alpha therapy for chronic hepatitis B. Lancet 343:244, 1994.

⁷⁴DiCesare E, Previti M, Russo F, Brancatelli S, Ingemi M C, Scoglio R, Mazzu N, Cucinotta D, Raimondo G: Interferon-alpha therapy may induce insulin autoantibody development in patients with chronic viral hepatitis. Digest Dis Sci 41:1672-77, 1996.

⁷⁵Waguri M, Hanafus T, Itoh N, Imagawa A, Miyagawa J, Kawata S, Kono N, Kuwajime M, Matsuzawa Y: Occurrence of IDDM during interferon therapy for chronic viral hepatitis. Diabetes Res Clin Pract 23:33-36, 1994.

⁷⁶Fabris P, Betterle C, Floreani A, Greggio N A, deLazzare F, Naccarato R, Chiaramonte M: Development of type 1 diabetes mellitus during interferon alpha therapy for chronic HCV hepatitis. Lancet 340:548, 1992.

⁷⁷Fattovich G, Giustine G, Favarato S, Ruol A: A survey of adverse events in 11,241 patients with chronic viral hepatitis treated with alpha interferon. J Hepatol 24:38-47, 1996.

⁷⁸Yamagishi S, Abe T, Sawada T: Human recombinant interferon alpha-2a (r INF alpha-2a) therapy suppresses hepatic triglyceride lipase, leading to severe hypertriglyceridemia in a diabetic patient. Am J Gastroenterol 89:2280, 1994.

⁷⁹Douvin C, Simon D, Charles M A, Deforges L, Bierling P, Lehner V, Buddowdka A, Dhumeaux D: Hepatitis B vaccination in diabeic patients: randomized trial comparing recombinant vaccines containing and not containing pre-S2 antigen. Diabetes Care 20:148-51, 1997.

⁸⁰Marseglia G L, Scaramuzza A, diAnnunzio G, Comolli G, Gatti G, Gatti M, Lorini R: Successful immune response to a recombinant hepatitis B vaccine in young patients with insulin-dependent diabetes mellitus. Diabetic Med 13:630-33, 1996.

⁸¹Ficicioglu C, Mikla S, Midilli K, Aydin A, Cam H, Ergin S: Reduced immune response to hepatitis B vaccine in children with insulin-dependent diabetes. Acta Paediatr Jpn 37:687-90, 1995.

⁸²Nolte W, Hartman H, Ramador G: Glucose metabolism and liver cirrhosis. Exp Clin Endocrinol Diabetes 103:63-74, 1995.

⁸³Petrides A S, Vogt C, Schulze-Berge D, Matthews D, Strohmeter G: Pathogenesis of glucose intolerance and diabetes mellitus in cirrhosis. Hepatology 19:616-27, 1994.

⁸⁴Sampelean D, Motocu M: Low insulin secretion in decompensated liver cirrhosis with diabetes mellitus. Rom J Intern Med 31:265-69, 1993.

⁸⁵Shah P, Mares D, Fineberg E, Pescovitz M, Filo R, Jindal R, Mahoney S, Lumeng L: Insulin autoimmune syndrome as a cause of spontaneous hypoglycemia in alcoholic cirrhosis. Gastroenerology 109:1673-76, 1995.

⁸⁶DeLissio M, Goodyear L J, Fuller S, Krawitt E L, Devlin J T: Effects of treadmill exercise on fuel metabolism in hepatic cirrhosis. J Appl Physiol 70:210-15, 1991.

⁸⁷Romijn J A, Endert E, Sauerwein H P: Glucose and fat metabolism during short-term starvation in cirrhosis. Gastroenterology 100:731-37, 1991.

⁸⁸Schneeweiss B, Pammer J, Ratheiser K, Schneider B, Madl C, Kramer L, Kranz A, Ferenci P, Druml W, Grimm G, Lera K, Garg A: Energy metabolism in acute hepatic failure. Gastroenterology 105:1515-21, 1993.

⁸⁹Eastman R C, Carson R E, Orloff D G, Cochran C S, Perdue J F, Rechler M M, Lanau F, Roberts C T Jr, Shapiro J, Roth J, Roith D: Glucose utilization in a patient with hepatoma and hypoglycemia: assessment by a positron emission tomography. J Clin Invest 89:1958-63, 1992.

⁹⁰Yonei Y, Tanaka M, Ozawa Y, Miyazaki K, Tsudada N, Inada S, Inagaki Y, Miyamoto K, Suziki O, Okawa H, Kibyu Y: Primary hepatocellular carcinoma with severe hypoglycemia: involvement of insulin-like growth factors. Liver 12:90-93, 1992.

⁹¹Barzilai N, Cohen P, Bar-Illan R, Mclntyre N, Karnieli E: Case report: increased insulin sensitivity in tumor hypoglycemia in a diabetic patient: glucose metabolism in tumor hypoglycemia. Am J Med Sci 302:229-34, 1991.

⁹²Ishida S, Noda M, Kuzuya N, Kubo F, Yamada S, Yamanaka T, Isozaki O, Hizuka N, Kanazawa Y: Big insulin-like growth factor II-producing hepatocellular carcinoma associated with hypoglycemia. Intern Med 34:1201-1206, 1995.

⁹³Hunter S J, Daughaday W H, Callender M E, McKnight J A, Mcilrath E M, Teale J D, Atkinson A B: A case of hepatoma associated with hypoglycaemia and overproduction of IGF-II (E21): beneficial effects of treatment with growth hormone and intrahepatic adriamycin. Clin Endocrinol Oxford 41:397-401, 1994.

⁹⁴Adami H O, Chow W H, Nyren O, Berne C, Linet M S, Ekbom A, Wolk A, McLaughlin J K, Fraumeni J F Jr: Excess risk of primary liver cancer in patients with diabetes mellitus. J Natl Cancer Inst 88:1472-77, 1996.

⁹⁵La Vecchia C, Negri E, Decarli A, Fanceschi S: Diabetes mellitus and the risk of primary liver cancer. Int J Cancer 73:204-207, 1997.

⁹⁶Fernandez O U, Canizares L L: Acute hepatotoxicity from ingestion of yellow phosphorus containing fireworks. J Clin Gastroenterol 21:139-42, 1995.

⁹⁷Kelly J H, Koussayer T, He D E, Chong M G, Shang T A, Whisennand H H, Sussman N L: An improved model of acetaminophen-induced fulminant hepatic failure in dogs. Hepatology 15:329-35, 1992.

⁹⁸Ilan Y, Shamir M, Eid A, Eidelman L, Tur-Kaspa R: Reversal of fulminant hepatitis associated hypoglycaemia at the anhepatic stage during liver transplantation. Neth J Med 48:185-87, 1996.

⁹⁹Carson K L, Hunt C M: Medical problems occurring after orthotopic liver transplantation. Digest Dis Sci 42:1666-74, 1997.

¹⁰⁰Trail K C, McCashland T M, Larsen J L, Jeffron T G, Strafta R J, Langnas A N, Fox I J, Zetterman R K, Donovan J P, Sorrell M F, Pillen T J, Ruby E I, Shaw B W Jr: Morbidity in patients with posttransplant diabetes mellitus following orthotopic liver transplantation. Liver Transplant Surg 2:276-83, 1996.

¹⁰¹Navasa M, Bustamante J, Marroni C, Gonzalez E, Andreu H, Esmatjes E, Garcia-Valdecasas J C, Grane L, Cirera I, Rimola A, Rodes J: Diabetes mellitus after liver transplantation: prevalence and predictive factors. J Hepatol 25:64-71, 1996.

¹⁰²Kanzler S, Lohse A W, Schirmacher P, Hermann E, Oto G, Meyer-zum-Buschenfelde K H: Complete reversal of FK 506 induced diabetes in a liver transplant recipient by change of immunosuppression to cyclosporine. Am J Gastroenterol 34:128-31, 1996.

¹⁰³Jin D C, Yoon Y S, Kim Y S, Yoon S A, Ahn S J, Kim S Y, Chang Y S, Bang B K, Koh Y B: Factors on graft survival of living donor kidney transplantation in a single center. Clin Transplant 10(6 pt 1):471-77, 1996.

¹⁰⁴Bonkovsky H L, Ponka P, Bacon B R, Drysdale J, Grace N D, Tavill A S: An update on iron metabolism: summary of the fifth international conference on disorders of iron metabolism. Hepatology 24:718-29, 1996.

¹⁰⁵Niederau C, Fischer R, Purschel A, Stremmel W, Haussinger D, Strohmeyer G: Long-term survival in patients with hereditary hemochromatosis. Gastroenterology 110:1107-19, 1996.

¹⁰⁶Hramiak I M, Finegood D T, Adams P C: Factors affecting glucose tolerance in hereditary hemochromatosis. Clin Invest Med 20:110-18, 1997.

¹⁰⁷Adams P C, Deugnier Y, Moirand R, Brissot P: The relationship between iron overload, clinical symptoms, and age in 410 patients with genetic hemochromatosis. Hepatology 25:162-66, 1997.

¹⁰⁸Turnbull A J, Mitchinson H C, Peaston R T, Lai L C, Bennett M K, Taylor R, Bassendine M F: The prevalence of hereditary hemochromatosis in a diabetic population. Quart J Med 90:271-75, 1997.

¹⁰⁹Chim C S, Chan V, Todd D: Hemosiderosis with diabetes mellitus in untransfused Hemoglobin H disease. Am J Hematol 57:160-63, 1998.

¹¹⁰Inoue Y, Nakanishi K, Hiraga T, Okubo M, Murase T, Kosaka K, Miyakoshi S, Mutoh Y, Kobayashi T: Recovery of pancreatic beta-cell function in hemochromatosis: combined treatment with recombinant human erythropoietin and phlebotomy. Am J Med Sci 314:401-402, 1997.

¹¹¹Ghishan F: Inborn errors of metabolism that lead to permanent liver injury. In Hepatology: A Textbook of Liver Disease. vol. II, 3rd edition. Zakim D and Boyer T D, Eds. Philadelphia, Pa., W. B. Saunders, 1996, 1574-1630.

¹¹²Ko G T, Szeto C C, Yeung V T, Chow C C, Chan H, Cockram C S: Autoimmune polyglandular syndrome and primary biliary cirrhosis. Br J Clin Pract 50:344-46, 1996.

hu 113Pohl R, Junge U: Primary sclerosing cholangitis with chronic pancreatitis. Dtsch Med Wochenschr 122:778-82, 1997.

¹¹⁴Kay M, Wyllle R, Michener W, Caulfield M, Steffen R: Associated ulcerative colitis, sclerosing cholangitis, and insulin-dependent diabetes mellitus. Cleve Clin J Med 60:473-78, 1993.

¹¹⁵lvarsson S A, Eridsson S, Kockum I, Lernmark A, Lindgren S, Nilsson K O, Sundkvist G, Wassmuth R: HLA-DR3, DQ2 homozygosity in two patients with insulin-dependent diabetes mellitus superimposed with ulcerative colitis and primary sclerosing cholangitis. J Intem Med 233:281-86, 1993.

¹¹⁶Kawashima C, Nagamine T, Takezawa J, Kon Y, Yamada S, Higuchi T, Mori M, Ohshima K: Studies on the glucose tolerance and the endocrine function of the pancreas in primary sclerosing cholangitis. Nippon Shokakibyo Gakkai Zaasshi 89:1425-32, 1992.

### LEGENDS TO FIGURES

FIG. 1 illustrates hepatic amino-N degradation (FHNC, litre/hour) for placebo and individuals treated with IGF-1 according to the invention.

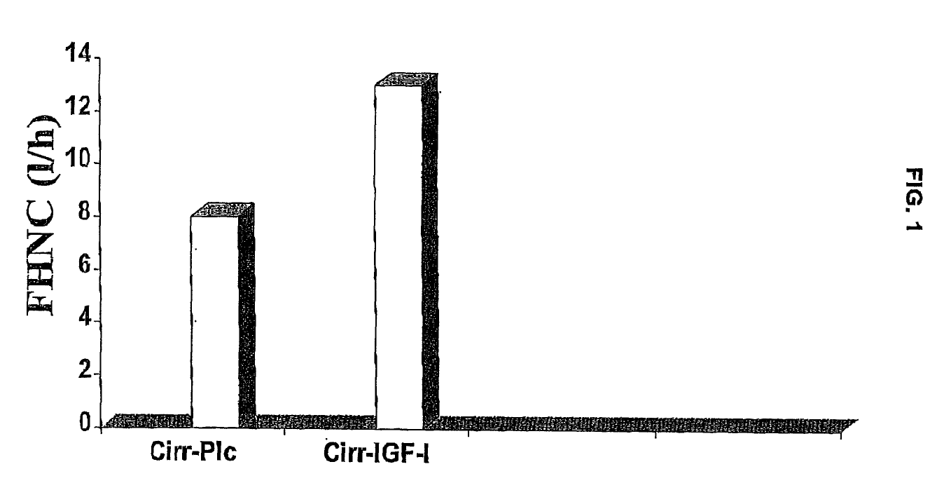

FIG. 2 illustrates insulin sensitivity (M-value) for placebo and individuals treated with IGF-1 according to the invention.

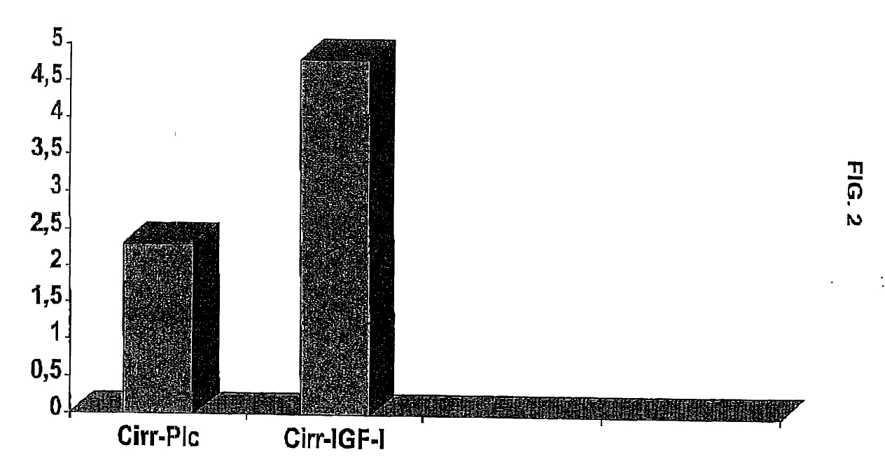

FIG. 3 illustrates selected biochemical values indicative of hepatic disease in 8 cirrhotic patients after 8 days of IGF-1 treatment.

FIG. 4 illustrates the glucose uptake across the human forearm in cirrhotic patients treated with IGF-1 or with placebo.

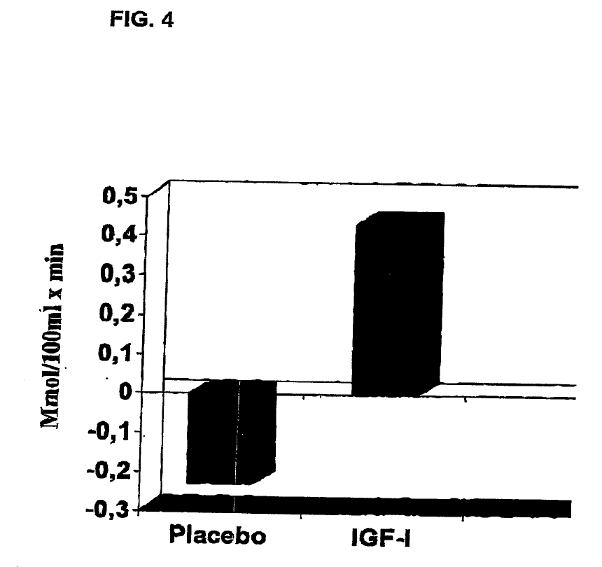

FIG. 5 illustrates the exchange of amino acids across the human forearm in cirrhotic patients treated with IGF-1 or with placebo.

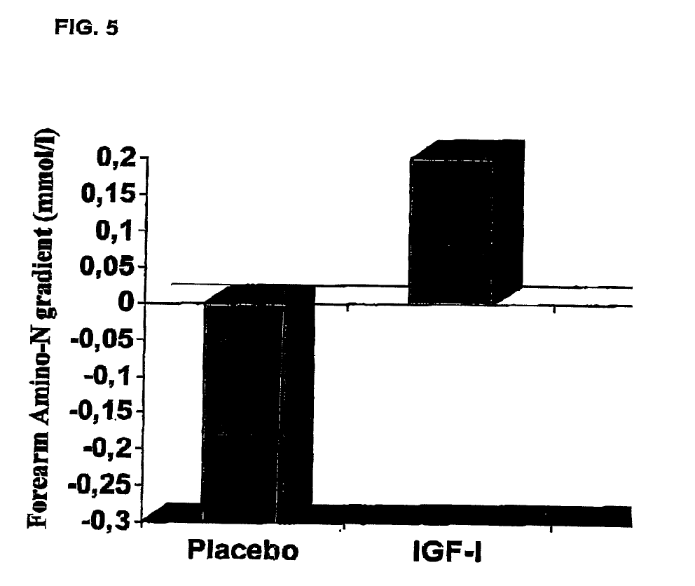

### EXAMPLES

The following examples demonstrate experimental procedures and preferred embodiments of the invention. The examples are illustrative only and should not be interpreted in any way that would confine the invention to the exact methods and results described therein.

### Example 1

Protocol

Energy Expenditure

Energy expenditure is assessed by indirect calometri before (−30 to 0) and just after (240-270) the gluc ose clamp. A comp uterized, open circuit system is employed to measure gas exchanges across a 25-L canopy (Deltatrac, Datex Instrumentarium Inc., Helsinki, Finland). The monitor determines carbon dioxide production and oxygen consumption by multiplying dry air flow through the canopy with the alterations in gas concentrations over the canopy.

Glucose Clamp Tegnique

After a baseline period, Insulin (Insulin Actrapid; Novo-Noridsk, Copenhagen, Denmark) was infused intravenously at a constant rate of 0.6 mU/kg/min for 180 min. Before baseline measurements, a bolus dose (17 μCi of [3-3H]glucose (DuPont-New England Nuclear, Boston, Mass., USA) was injected, followed by a constant rate infusion (0.17 μCi/min) throughout the experiment. Plasma glucose was clamped at 5 mmol/l as described by DeFronzo et al. In order to minimize rapid dilution of the labelled glucose pool with uniabelled glucose, [3-3H]glucose was added to the glucose infused during the clamp.

FHNC

To estimate hepatic amino acid metabolism urea nitrogen synthesis rate (UNSR) and blood alfa-amino nitrogen levels is measured before, during and after a 4 h constant iv infusion of alanine (2 mmol/kg BW×h). UNSR was estimated hourly as urinary excretion corrected for accumulation in body water. The slope of the linear relationship between UNSR and circulating alanine levels represents the hepatic components of conversion of amino nitrogen and is denoted the functional hepatic nitrogen clearance (FHNC).

Alanine (2 mmol/kg bodyweight) (Ajinomoto Co. Inc., Tokyo, Japan), given by volumetric pump (Terufusion STC-503, Rødovre, Denmark) through a catheter inserted in an antecubutal vein, was used to stimulate amino nitrogen conversion as measured by the FHNC. Alanine infusions were initiated at 0800 h and continued for 3 h, implying that the 3 h(from 0800-1100 h) with increasing amino acid concentrations and the following 2 h(from 1100-1300 h) with decreasing amino acid concentrations could be incorporated in the calculation of FHNC.

The urea nitrogen synthesis rate(UNSR) (mmol/h) was calculated as urinary excretion rate (E), corrected for accumulation (A) in total body water (TBW) and for the fractional intestinal loss (L):

UNSR=(E+A)/(1−L),

where E=(urine flow, l/h)×(urinary urea-N, mmol/h), A=(change in blood urea-N, mmol/(l×h))×(TBW, litre).

L was taken to be 0.14 {157}.

TBW was assessed from body weight (BW, kg), body height (BH, cm) and age (Y, years), by the formula {157}:

TBW=0.3625×BW+0.2239×BH−0.1387×Y−14.47 for men

Body weight did not change during or between investigations, so it was assumed that TBW also remained stable.

FHNC (l/h) was calculated as the slope of the linear regression analysis of UNSR on corresponding mean blood a-amino nitrogen concentrations. This measure standardises urea nitrogen synthesis rate with regard to changes in blood a-amino nitrogen concentration. Six data sets were available for each determination.

Forearm Substrate Exchange

Catheters (Venflon, Viggo, Helsingborg, Sweden) for measurements of forearm arterio-deep venous substrate balances were placed as described previously{724}: At 0630 h a catheter was inserted retrogradely into a deep antecubital vein of one arm for sampling blood derived from the forearm muscles.

The criteria for satisfactory positioning were that the tip of the catheter could not be palpated, and that the oxygen saturation in blood drawn from the catheter was below 70%. In the contralateral arm, one catheter was placed retrogradely in a heated dorsal hand vein for sampling of arterialized blood.

Oxygen saturation in the blood drawn from the heated dorsal vein was consistently above 92%. Finally one catheter was placed in an antecubital vein of the heated arm for all infusions.

In each individual the same veins were used for insertion on each occasion. Preceding every deep venous sample, total ipsilateral forearm blood flow is determined by means of venous occlusion plethysmography. Hand blod flow is interrupted by a wrist cuff inflated to a pressure of 250 mm Hg immediately before every blood flow determination and 1 min before every deep venous blood sample.

Substrate balances across the deep forearm tissues are calculated as the production of blood flow (milliliters per 100 mL tissue/min) to the tissues drained by the deep forearm vein and change in the total blood content (arterialized blood minus deep venous blood) of each metabolite across the forearm. For these calculations it is assumed that the relative blood flow (milliliters per 100 mL/min) to the tissues drained by the deep forearm vein equals 0.47×total forearm blood flow+0.83.

Investigations

In each investigation urea nitrogen synthesis rate (UNSR) and blood a-amino nitrogen concentration were measured in 6 consecutive 60 min intervals (from −60 to 300 min).

Blood samples were drawn from the catheter inserted in the heated dorsal hand vein. Blood concentrations of urea nitrogen and a-amino nitrogen were measured at time −60, −45, −15 and 0 the first hour, and every 60 minutes for the rest of the experiment; a-amino nitrogen represents the sum of all amino acids.

Blood samples for measurements of serum-insulin, C-peptide, growth hormone, GHBP, IGFBP's, NEFA, 3-hydroxybutyrate, glycerol, lactate, alanine, glucose, glucagons and glucose specific activity were taken at time −60, and every hour through the rest of the experiments. Samples were immediately frozen after centrifugation at −80° C. until assayed. The hourly blood samples were obtained with exact time registration, immediately after voiding each one hour urine sample (see below).

Subjects drank a minimum of 200 ml tapwater pr. hour to keep urine production above 120 ml/h. The bladder was emptied by voiding at 60 min intervals, urine volumes were measured and samples frozen for later determination of urea nitrogen concentration.

Preceding every deep venous sample, total ipsilateral forearm blood flow was determined by means of venous occlusion plethysmography{724}. Hand blod flow was interrupted by a wrist cuff inflated to a pressure of 250 mm Hg immediately before every blood flow determination and 1 min before every deep venous blood sample. Arterialized and deep venous blood samples were drawn simultaneously at the following time points: −60, 0, 120, 240, 360

Analyses

Urea nitrogen concentration in urine and blood was measured by the urease-Berthelot method {191}.

Blood a-amino nitrogen concentration by the dinitroflourobenzene method {192}.

Serum insulin concentrations were measured in duplicate by a two-site immunospecific insulin ELISA.

Plasma glucagon concentrations were measured using radioimmunoassays as described by Ørskov et al. {783}

Growth hormone concentrations were determined by radioimmunoassays (DELFIA, Wallac, Finland).

Plasma glucose were analysed in duplicate by use of a Beckman glucoanalyzer immediately after sampling (Beckman instruments, Palo Alto, Calif., USA). After counting the plasma specific activity of glucose the non-steady state equation of Steele as modified by DeBodo et al. was used for calculation of glucose appearance/disposal rates. A pool fraction of 0.65 and a distribution volume of 220 ml/kg were assumed.

Respiratory exchange ratios were determined by indirect kaliometri.

Protein oxidation rates were estimated from urinary excretion of urea.

Net lipid oxidation and glucose oxidation rates were computed from the above measurements, and non-oxidative glucose disposal was calculated by subtracting the glucose oxidation rates from total isotopically determined glucose disposal.

Results

Glucose and Hormones

IGF-I concentrations increased 5 fold after treatment (48±5 vs 260±25 ng/ml, p<0.05).

Basal plasma glucose concentrations were significantly higher in control situation compared to IGF-I treatment (112±5 mg/100 ml vs 96±5 mg/100 ml, p<0.05).

Serum insulin concentrations during baseline were more than twice as high in placebo compared to IGF-I (98±8 pmol/l vs 41±6 pmol/l, p<0.05), and serum C-peptide followed the same pattern.

Basal hepatic glucose production (HGP) decreased by 25% after IGF-I (2.91±0,1 vs 2.11±0.1 mg/kg/min, p<0.05), and by 70% during hyperinsulinaemia ((0.81±0.1 vs 0.19±0.1 mg/kg/min, p<0.05).

Basal rates of glucose disposal increased by 30% after IGF-I (1.41±0.1 vs 2.01±0.1 mg/kg/min, p<0.05).

The ability of insulin to stimulate whole-body glucose disposal was also significantly increased after IGF-I treatment (2.31±0.3 vs 4.81±0.45 mg/kg/min, p<0.05). Both basal and insulin stimulated glucose oxidation were increased after IGF-I treatment.

Non-oxidative glucose disposal were markedly increased after IGF-I, suggesting that part of the increased glucose utilisation was caused by increased glycogen formation in muscles and part of it as glycogen synthsized in the liver. All in all this means that glucose metabolism was markedly improved after treatment with IGF-I.

Glucagon decreased by 33% after IGF-I (130±12 vs 87±11 pg/ml, p<0.05).

Growth hormone decreased almost 3 fold after IGF-I (3.1±1.2 vs 1.31±0.5, p<0.05). IGFBP-I doubled after IGF-I treatment, probably due to the decrease in insulin levels (12 vs 23?, p<0.05). IGFBP-3 and GHBP did not change.

Functional Hepatic Nitrogen Clearance (FHNC)

Baseline blood a-amino-N concentrations were slightly higher in the placebo situation compared to the IGF-I situation When alanine was infused in the control experiment there was a gradual increase to a maximum of 7.4±0.3 mmol/l at 240 min. After IGF-I treatment the rise was less pronounced with a maximum of 5.9±0.17 mmol/l, (p<0.05 by Students t-test).

According to the same pattern, infusion of alanine gradually increased UNSR to a different maximum value of 100±6 mmol/h during placebo treatment and to 123±13 mmol/h during IGF-I treatment (p<0.05).

The functional hepatic nitrogen clearance (FHNC) increased on average by 30% after treatment with IGF-I, meaning that the hepatic efficacy for amino acid disposal was markedly improved in cirrhotic patients treated with IGF-I. Potentially, this could avoid the development of hepatic encephalopathy in advanced liver disease.

### Example 2

Insulin-Like Growth Factor-1 Improves Glucose Metabolism and Hepatic Amino Acid-N Conversion in Patients with Liver Cirrhosis

Reduced bioavailability of insulin-like growth factor-1 (IGF-1) together with disturbances in intermediary glucose and amino acid metabolism are common features in patients with liver cirrhosis. One objective of the present invention was to compare the effect of IGF-1 with placebo on glucose and amino acid metabolism in a randomly sequenced cross over design.

6 patients with alchoholic liver cirrhosis were investigated twice after 7 days placebo (vehicle) and after 7 days with IGF-1 (0.1 mg/kg/day). The functional hepatic nitrogen clearance (FHNC) which describes substrate independent changes in hepatic amino acid conversion and the hyperinsulinaemic euglycemic clamp (insulin infusion rate: 0.6 mU/kg min for 180 min) to determine insulin sensitivity were performed.

Results

IGF-1 treatment increased both FHNC (13.3±4 compared to placebo: 7.7±4 l/h, p<0.01) and insulin-stimulated glucose disposal (4.8±0.4 compared to 2,1±0.4 mg/kg min) markedly. Hepatic glucose output ([3-3H]glucose) was nearly halved and basal levels of insulin, C-peptide, glucagon and growth hormone decreased during IGF-1 treatment.

Conclusions

IGF-1 increases hepatic amino acid-N conversion and improves glucose disposal in cirrhotic patients. The amelioration of these fundamental metabolic consequenses of cirrhosis has implications for the pathophysiological understanding of impairment of liver function, and may indicate new treatment principles of cirrhosis.

### Example 3

Improved Forearm Substrate Exchange and Biochemical Values in Cirrhotic Patients After Treatment with IGF-1

8 patients suffering from cirrhosis classified as either Child's class A or B were treated for 8 days with placebo (vehicle) or with IGF-1 (0.1 mg/kg/day).

After 8 days of treatment individuals were tested for their biochemical values indicative of hepatic disease as well as for forearm substrate exchange rates as described in example 1. In particular the glucose exchange and amino-N exchange were deternined as well as the biochemical values in blood samples for albumin, coagulation factor 3, 7 and 10 (INR), alanine aminotransferase (ALAT), aspartate amino-transferase (ASAT) and γ-glutamyl transferase (GGT).

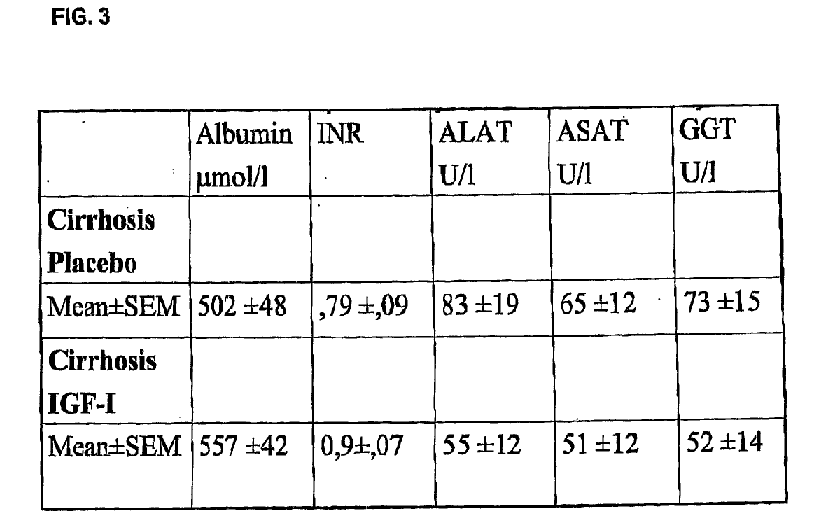

After 8 days of IGF-1 treatment the biochemical values for albumin, INR, ALAT, ASAT and GGT, which are all indicative of hepatic disease were improved although not significantly (FIG. 3).

IGF-1 treatment increased the glucose uptake across the human forearm almost 3 fold compared to placebo in the basal state (see FIG. 3).

Furthemore, IGF-1 treatment increased the exchange of amino acids across the human forearm almost 2 fold compared to placebo. These results indicates beneficial effects of IGF-1 treatment on muscle protein build up.

All patents, patent applications and non patent references cited anywhere in this specification, including but not limited to Ser. No. 60/237,715 and DK PA 2000 01317, are hereby incorporated by reference in their entirety.

1. Method of treatment of an individual suffering from a liver disease, or at risk of contracting a liver disease unless treated, said method comprising the step of administering to the individual a composition comprising a pharmaceutically effective amount of IGF-1, or a variant thereof.

2. Method of claim 1, wherein said individual is a human being.

3. Method of claim 1, wherein said liver disease is acute liver disease.

4. Method of claim 3, wherein said liver disease is liver failure.

5. Method of claim 3, wherein said liver disease occurs in combination with malnutrition.

6. Method of claim 3, wherein said liver disease occurs in combination with insulin resistance.

7. Method of claim 3, wherein said liver disease occurs in combination with IGF-1 deficiency.

8. Method of claim 1, wherein said liver disease is chronic liver disease.

9. Method of claim 8, wherein said liver disease is cirrhosis of the liver.

10. Method of claim 8, wherein said liver disease is fibrosis of the liver.

11. Method of claim 8, wherein said liver disease is chronic hepatitis.

12. Method of claim 8, wherein said liver disease occurs in combination with a metabolic disorder.

13. Method of claim 8, wherein said liver disease occurs in combination with malnutrition.

14. Method of claim 8, wherein said liver disease occurs in combination with insulin resistance.

15. Method of claim 8, wherein said liver disease occurs in combination with diabetes mellitus.

16. Method of claim 8, wherein said liver disease occurs in combination with IGF-1 deficiency.

17. Method of claim 1, wherein said pharmaceutically effective amount of IGF-1 is less than 200 microgram and more than 25 microgram per day per kilo gram of treated individual.

18. Method of claim 1, wherein said pharmaceutically effective amount of IGF-1 is about 100 microgram per day per kilogram of treated individual.

19. Method of claim 1, wherein the IGF-1 is recombinant IGF-1, or a variant thereof, produced by recombinant DNA technology.

20. Method of claim 1, wherein said composition further comprises at least one IGF-1 binding protein (IGFBP) selected from the group consisting of IGFBP-1, IGFBP-2, IGFBP-3, IGFBP-4, IGFBP-5, and IGFBP-6, and variants thereof.

21. Method of claim 2, wherein said composition further comprises at least one IGF-1 binding protein (IGFBP) selected from the group consisting of IGFBP-1, IGFBP-2, IGFBP-3, IGFBP-4, IGFBP-5, and IGFBP-6, and variants thereof.

22. Method of claim 9, wherein said composition further comprises at least one IGF-1 binding protein (IGFBP) selected from the group consisting of IGFBP-1, IGFBP-2, IGFBP-3, IGFBP-4, IGFBP-5, and IGFBP-6, and variants thereof.
